(19) United States
(12) Patent Application Publication
(10) Pub. No.: US 2023/0056977 A1
(43) Pub. Date: Feb. 23, 2023

(54) POSTURE DETECTION SYSTEM AND POSTURE DETECTION METHOD

(71) Applicants: Karlos ISHAC, ROZELLE (AU); Katia BOURAHMOUNE, ROZELLE (AU)

(72) Inventors: Karlos ISHAC, ROZELLE (AU); Katia BOURAHMOUNE, ROZELLE (AU)

(21) Appl. No.: 17/796,600

(22) PCT Filed: Jan. 31, 2020
(86) PCT No.: PCT/JP2020/003783

§ 371 Date: Jul. 29, 2022

(51) Int. Cl. A61B 5/11; G01L 5/162; B60N 2/00; B60N 2/90; A61B 5/00; A63B 24/00; A61B 5/16

(52) U.S. Cl. CPC A61B 5/1116; G01L 5/162; B60N 2/002; B60N 2/90; A61B 5/6891; A61B 5/7455; A61B 5/7267; A61B 5/486; A61B 5/742; A63B 24/0075; A61B 5/746; A61B 5/165; B60N 2002/981; A61B 2562/0247

(57) ABSTRACT

A posture detection system for detecting a user's posture according to the embodiments includes a pressure sensor unit, a controller, a feedback mechanism, and a display unit. The pressure sensor unit has a sheet shape or a padded shape and includes plurality of sensors. Each of the sensors is configured to detect a pressure applied from the user. The controller is configured to classify the user's posture based on detection data detected by the pressure sensor unit. The feedback mechanism is configured to provide feedback to the user by vibrating based on a result of the classification. The display unit is configured to perform a display according to the result of the classification.

### TECHNICAL FIELD

The present disclosure relates to a posture detection system and a posture detection method.

### BACKGROUND ART

Patent Literature 1 discloses an apparatus for detecting a user's sitting posture. An array of pressure sensor pads is embedded in a backrest cushion of this apparatus. The apparatus includes an algorithm for classifying sitting postures according to a result of the detection on the pressure sensor pads. The apparatus includes straps to attach a cushion to a chair.

### CITATION LIST

### Patent Literature

Patent Literature 1: Australian Patent Application Publication No. 2017101323

### SUMMARY OF INVENTION

### Technical Problem

Such an apparatus is desired to detect a posture more appropriately and provide feedback effectively.

This embodiment has been made in view of the above point. An object of this embodiment is to provide a posture detection system and a posture detection method that can appropriately detect a posture and provide feedback effectively.

### Solution to Problem

A posture detection system according to the embodiment including: a pressure sensor unit including plurality of sensors, each of the sensors being configured to detect a pressure applied from the user; a controller configured to classify the user's posture based on detection data detected by the pressure sensor unit; a feedback mechanism configured to provide feedback to the user by vibrating based on a result of the classification; and a display unit configured to perform a display according to the result of the classification.

### Advantageous Effects of Invention

According to this embodiment, it is possible to provide a posture detection system and a posture detection method that can appropriately detect a posture and provide feedback effectively.

### BRIEF DESCRIPTION OF DRAWINGS

FIG. 1 shows a main part of a posture detection system;

FIG. 2 shows a backrest cushion of the posture detection system according to this embodiment;

FIG. 3 shows the backrest cushion of the posture detection system according to this embodiment;

FIG. 4 is a front view showing an arrangement of sensors and vibrators in the backrest cushion;

FIG. 5 is a front view showing an arrangement of sensors and vibrators in a seating face sensor unit;

FIG. 6 is an exploded perspective view showing a layered configuration of a pressure sensor unit;

FIG. 7 is a side cross-sectional view showing an example of the layered configuration of the pressure sensor unit;

FIG. 8 is a side cross-sectional view showing an example of the layered configuration of the pressure sensor unit;

FIG. 9 is a drawing showing a control system of the posture detection system;

FIG. 10 is a flowchart showing a posture detection method;

FIG. 11 is a drawing showing an example of a table for classifying postures;

FIG. 12 is a drawing showing another example of a table for classifying postures;

FIG. 13 is a flowchart showing a method for providing haptic feedback;

FIG. 14 is a drawing for describing a configuration for measuring vital information using a vibration sensor;

FIG. 15 is a drawing for describing a difference in measurement signals according to breathing timings;

FIG. 16 is a flowchart for describing processing for determining a user's fatigue level;

FIG. 17 is a table showing classification of driver states;

FIG. 18 is a table showing classification of driving actions;

FIG. 19 is a flowchart showing processing for outputting an alert or a reminder to a user;

FIG. 20 is a flowchart showing processing in a stretching guidance mode;

FIG. 21 is a drawing showing a pressure distribution in each stretch pose;

FIG. 22 is a flowchart showing processing in a meditation guidance mode;

FIG. 23 is a flowchart for describing processing for reducing pain;

FIG. 24 is a drawing showing a posture detection system including an exercise member;

FIG. 25 is a drawing showing a display example of a health care report;

FIG. 26 is a flowchart showing processing for creating a health care report;

FIG. 27 is a flowchart showing processing for classifying postures using a learned model;

FIG. 28 is a flowchart showing processing for predicting a user's behavior using a learned model;

FIG. 29 is a flowchart showing processing for classifying a fatigue level using a learned model;

FIG. 30 is a flowchart showing processing for identifying a user using a learned model;

FIG. 31 is a drawing showing an example in which a pressure sensor sheet is mounted on a wheelchair;

FIG. 32 is a drawing showing an example in which the pressure sensor sheet is mounted on the wheelchair; and

FIG. 33 is a drawing showing an example in which a pressure sensor sheet is mounted on a driver's seat of a vehicle.

### DESCRIPTION OF EMBODIMENTS

Hereinafter, specific embodiments to which the present disclosure is applied will be described in detail with reference to the drawings. However, the present disclosure is not limited to the following embodiments. Note that the following description and drawings are simplified as appropriate in order to clarify the descriptions.

### First Embodiment

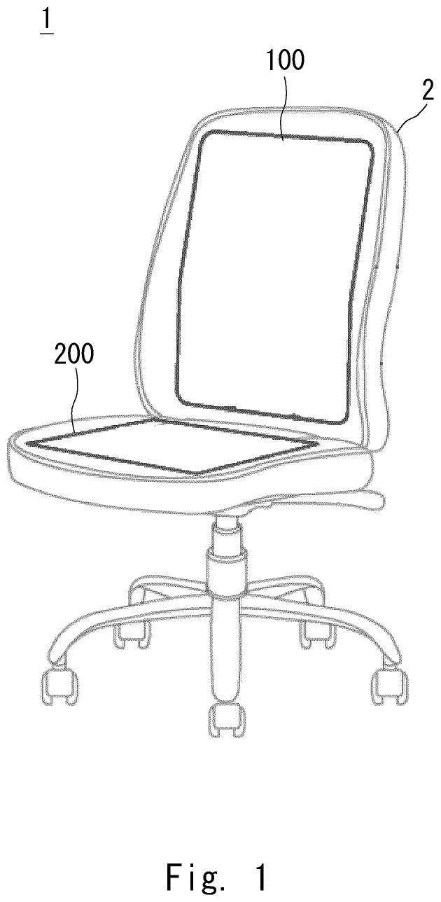

A posture detection system and method according to this embodiment will be described with reference to the drawings. FIG. 1 shows a main part of the posture detection system 1. The posture detection system 1 includes a backrest cushion 100 and a seating face cushion 200. The backrest cushion 100 is attached to a backrest of a chair 2. The seating face cushion 200 is attached to a seating face of the chair 2. In the following description, the front-rear direction, the left and right direction, and the vertical direction are directions viewed from a user sitting on the chair 2.

In FIG. 1, the posture detection system 1 is attached to a chair in, for example, an office. However, the posture detection system 1 may be attached to, for example, a wheelchair seat and a driver's seat. The posture detection system 1 may be provided in the driver's seat and a boarding seat of a conveyance such as an automobile, a vehicle, a train, and an airplane.

The backrest cushion 100 is placed on the user's back side. A pressure sensor unit described later is built into the backrest cushion 100. The seating face cushion 200 is placed under the user's bottom. A seating face sensor unit described later is built into the seating face cushion 200.

Each of the backrest cushion 100 and the seating face cushion 200 detects a pressure applied by the user. The backrest cushion 100 and the seating face cushion 200 are detachable from the chair 2. The backrest cushion 100 and the seating face cushion 200 do not need to be detachable from the chair 2. That is, the backrest cushion 100 may be incorporated as a backrest of the chair 2, and the seating face cushion 200 may be incorporated as a seating face of the chair 2.

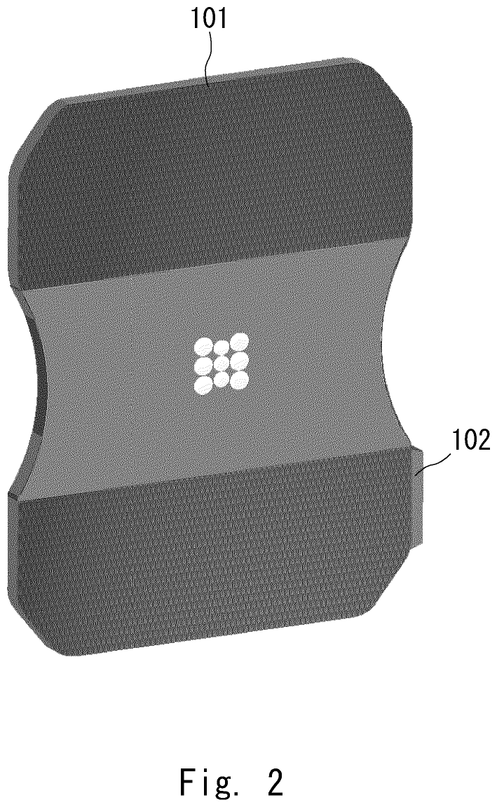

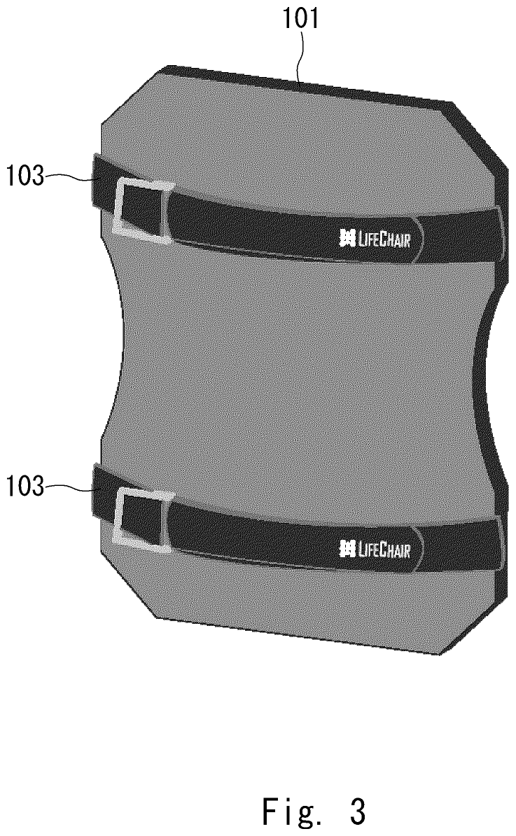

FIGS. 2 and 3 are perspective views showing a configuration of the backrest cushion 100. FIG. 2 shows the backrest cushion 100 as viewed from the front side, and FIG. 3 shows the backrest cushion 100 as viewed from the back side. That is, FIG. 2 shows a contact surface of the backrest cushion 100 that is brought into contact with the user's back, and FIG. 3 shows a surface opposite to the contact surface.

The backrest cushion 100 includes a cushion part 101, a control module 102, and belts 103. A pressure from the user's back is applied to the cushion part 101. A pressure sensor unit provided in the cushion part 101 detects the pressure.

The belts 103 are provided on the back side of the cushion part 101. Here, two belts 103 are attached to the cushion part 101. The number of belts 103 may be one, or three or more, as a matter of course. One ends of the belts 103 are attached to the left end of the cushion part 101, and the other ends of the belts 103 are attached to the right end of the cushion part 101. By placing the backrest of the chair 2 is placed between the cushion part 101 and the belts 103, the backrest cushion 100 is attached to the chair 2. The belts 103 may be formed of an elastic body such as rubber. Note that, when the backrest cushion 100 is fixed to the chair 2, the belts 103 are not necessary.

The control module 102 is provided on the side surface of the cushion part 101. The control module 102 includes a processor, a memory, etc. The control module 102 further includes a power button, a power indicator light, a charging port, and so on. By pressing the power button, the power indicator light is turned on and the posture detection system 1 operates. For example, a USB port is used as the charging port. That is, the battery built into the cushion part 101 is charged by inserting a USB cable into the port.

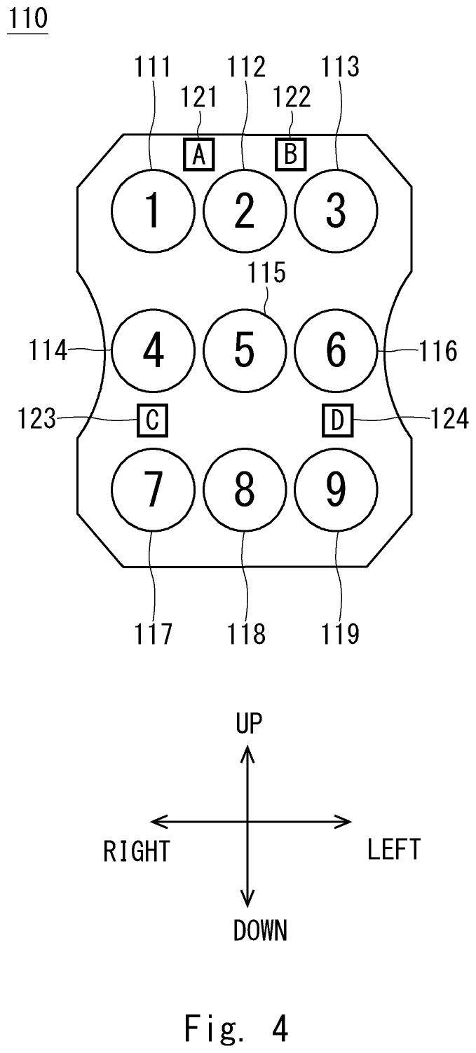

FIG. 4 shows the pressure sensor unit and vibrators provided in the cushion part 101. FIG. 4 shows a pressure sensor unit 110 as viewed from the front. The pressure sensor unit 110 includes a plurality of sensors 111 to 119. Here, the pressure sensor unit 110 includes nine sensors 111 to 119. The sensors 111 to 119 are arranged in a 3x3 array. Each of the sensors 111 to 119 is connected to the control module 102 via wiring. Each of the sensors 111 to 119 outputs a detection signal corresponding to the detected pressure to the control module 102.

The sensors 111 to 113 are arranged in the upper row, the sensors 114 to 116 are arranged in the middle row, and the sensors 117 to 119 are arranged in the lower row. The sensors 111, 114, and 117 are arranged on the right side of the user, and sensors 113, 116, and 119 are arranged on the left side of the user. The sensors 112, 115, and 118 are arranged at the center of the user in the left and right direction. The positions of sensors 111 to 119 are defined as position 1 to position 9, respectively. For example, the position of the sensor 111 is the position 1. The size and arrangement of the sensors 111 to 119 may be the same as those of Patent Literature 1. Obviously, the arrangement and number of sensors 111 to 119 are not limited to the configuration shown in the drawings.

The cushion part 101 further includes vibrators 121 to 124. Each of the vbrators 121 to 124 includes an electric motor, a piezoelectric element, etc. Each of the vibrators 121 to 124 is connected to the control module 102 via wiring. The vibrators 121 to 124 vibrate in accordance with control signals from the control module 102.

The vibrators 121 and 122 are placed above the sensors 111 to 113. The vibrator 123 is placed between the sensors 114 and 117. That is, the vibrator 123 is placed below the sensor 114 and above the sensor 117. The vibrator 123 is placed below the sensor 114 and above the sensor 117. The positions of the vibrators 121 to 124 are defined as positions A to D, respectively. For example, the position of the vibrator 121 is the position A.

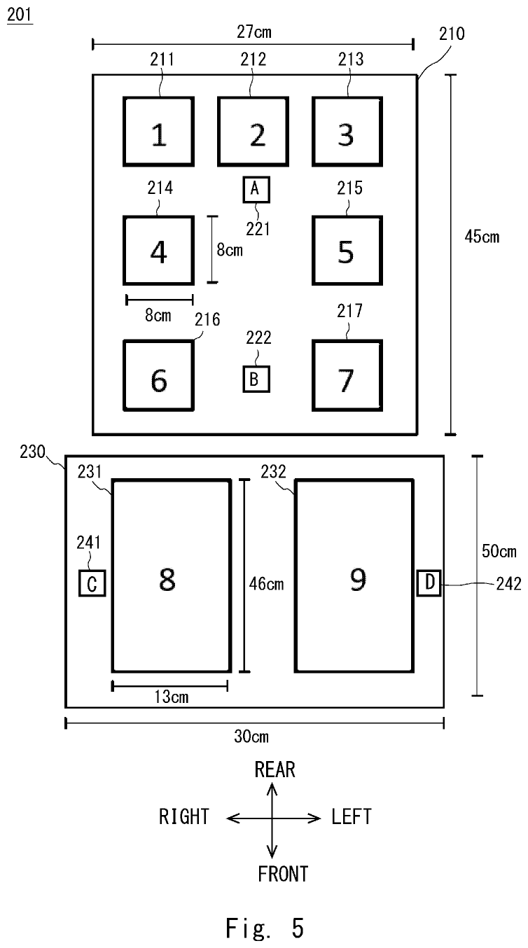

FIG. 5 shows an arrangement example of a seating face sensor unit 201 provided in the seating face cushion 200. The seating face sensor unit 201 includes a first seating face sensor sheet 210 and a second seating face sensor sheet 230. The second seating face sensor sheet 230 is placed before the first seating face sensor sheet 210. For example, the first seating face sensor sheet 210 is placed under the user's bottom, and the second seating face sensor sheet 230 is placed under the user's thighs.

The first seating face sensor sheet 210 includes a plurality of sensors 211 to 217. Here, seven sensors 211 to 217 are provided on the first seating face sensor sheet 210. The sensors 211 to 213 are placed on the rear side the first seat sensor sheet 210, and the sensors 216 and 217 are placed on the front side of the first seating face sensor sheet 210. The positions of the sensors 211 to 217 are defined as positions 1 to 7, respectively. For example, the position of the sensor 211 is the position 1. Each of the sensors 211 to 217 has a square shape of 8 cm×8 cm.

Furthermore, the first seating face sensor sheet 210 includes a plurality of vibrators 221 and 222. Here, two vibrators 221 and 222 are provided on the first seating face sensor sheet 210. The vibrators 221 and 222 are placed at the center of the first seating face sensor sheet 210 in the left and right direction. The vibrators 221 and 222 are placed on the front side of the sensor 212. The position of the vibrator 221 is defined as a position A, and the position of the vibrator 222 is defined as a position B.

The second seating face sensor sheet 230 includes a plurality of sensors 231 and 232. Here, two sensors 231 and 232 are provided on the second seating face sensor sheet 230. The sensor 231 is placed on the right side of the second seating face sensor sheet 230, and the sensor 232 is placed on the left side of the second seating face sensor sheet 230. For example, the sensor 231 is placed under the user's right thigh, and the sensor 232 is placed under the user's left thigh. The position of the sensor 231 is defined as a position 8, and the position of the sensor 232 is defined as a position 9.

Furthermore, the second seating face sensor sheet 230 includes a plurality of vibrators 241 and 242. Here, two vibrators 241 and 242 are provided on the second seating face sensor sheet 230. The vibrator 241 is placed on the right side of the sensor 231, and the vibrator 242 is placed on the left side of the sensor 232. The position of the vibrator 241 is defined as a position C, and the position of the vibrator 242 is defined as a position D.

Note that the positions, numbers, arrangements, and shapes of the sensors and vibrators are examples of this embodiment, and are not limited to those described above. The seating face sensor unit 201 may have either of the first seating face sensor sheet 210 or the second seating face sensor sheet 230. For example, the second seating face sensor sheet 230 is optional and can be omitted. That is, the seating face sensor unit 201 has only the first seating face sensor sheet 210. Or, the first seating face sensor sheet 210 is optional and can be omitted. That is, the seating face sensor unit 201 has only the second seating face sensor sheet 230.

The posture detection system 1 may have either of the seating face sensor unit 201 or the pressure sensor sheet 110. For example, the pressure sensor sheet 110 is optional and can be omitted. That is, the posture detection system 1 has only the seating face sensor unit 201. Or, the seating face sensor unit 201 is optional and can be omitted. That is, The posture detection system 1 has only the pressure sensor sheet 110.

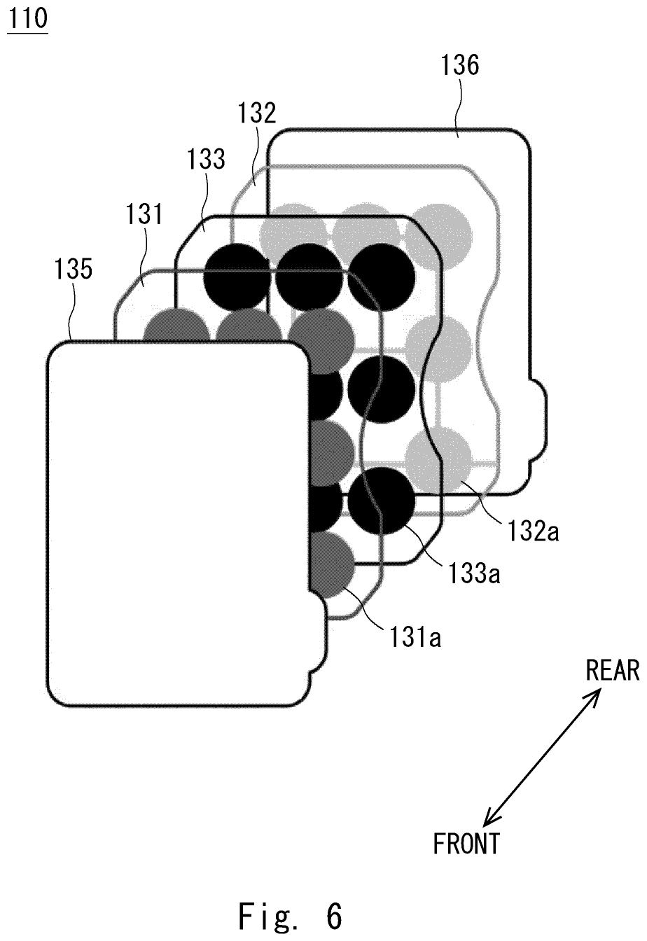

The pressure sensor unit 110 is formed in a sheet shape or a padded shape. The pressure sensor unit 110 may be attached to wheel chair or seat. The pressure sensor unit 110 may be just placed on the back or bottom of the user. The pressure sensor unit 110 may be built into a chair and so on. The pressure sensor unit 110 or the seating face sensor unit 201 may be a single cushion. Alternatively, the pressure sensor unit 110 or the seating face sensor unit 201 may be directly embedded into the chair. The pressure sensor unit 110 has a layered structure in which a plurality of layers are stacked. The layered structure of the pressure sensor unit 110 will be described with reference to FIG. 6. FIG. 6 is an exploded perspective view of the pressure sensor unit 110.

The pressure sensor unit 110 includes a first layer 131, a second layer 132, a third layer 133, a front cover layer 135, and a back cover layer 136. The back cover layer 136, the second layer 132, the third layer 133, the first layer 131, and the front cover layer 135 are placed in this order from the rear side of the user toward the front (user's back side).

The first layer 131 includes a plurality of sensing electrodes 131a. The sensing electrodes 131a correspond to the sensors 111 to 119 shown in FIG. 4, respectively. Nine sensing electrodes 131a are provided on the first layer 131. The nine sensing electrodes 131a are independent from each other. Each of the sensing electrodes 131a is connected to the circuit of the control module 102 by independent wiring. The sensing electrodes 131a are formed of conductive fabric. For example, each of the sensing electrodes 131a is formed by trimming the conductive fabric into the shape of a circle. The thickness of the first layer 131 is, for example, 0.05 mm to 0.30 mm. The sensing electrode 131a may be formed of conductive tape, instead of the conductive fabric. For example, The sensing electrode 131a may be formed of adhesive copper tape.

The second layer 132 is formed of a conductive sheet 132a with variable resistance. The second layer 132 is placed between the first layer 131 and the third layer 133. That is, a front surface of the second layer 132 is brought into contact with the first layer 131 and a back surface of the second layer 132 is brought into contact with the third layer 133. The second layer 132 is formed of a sheet such as velostat or polymeric foil. Thus, an electrical resistance of the conductive sheet 132a changes according to the pressure received by each of the sensors 111 to 119. The thickness of the second layer 132 is, for example, 0.05 mm to 0.30 mm. The second layer 132 may be a piezoresistice sheet. For example. the the second layer 132 may be formed by a single sheet of couductive film (a piezoresistive sheet) that covers the surface area of the first layer 131.

The conductive sheet 132a overlaps the sensing electrodes 131a. In FIG. 6, the conductive sheet 132a is separated in such a way that separated pieces of the conductivge sheet 132a face the respective sensing electrodes 131a. That is, nine pieces of conductive sheet 132a each having the same size as that of the sensing electrode 131a are prepared and placed so as to face the respective sensing electrodes 131a. Alternatively, a single large conductive sheet may be used. That is, one conductive sheet such as the piezoresistive sheet may cover the nine sensing electrodes 131a.

The third layer 133 is placed behind the second layer 132. The third layer 133 includes counter electrodes 133a facing the sensing electrodes 131a. That is, the sensing electrodes 131a and the counter electrodes 133a are placed to face each other with the conductive sheet 132a interposed therebetween. The third layer 133 includes nine counter electrodes 133a. Each of the counter electrodes 133a may have the same size as that of the sensing electrode 131a or a size different from that of the sensing electrode 131a.

The counter electrodes 133a are formed of conductive fabric. For example, each of the counter electrodes 133a is formed by trimming the conductive fabric into the shape of a circle. The thickness of the third layer 133 is, for example, 0.05 mm to 0.30 mm. The nine counter electrodes 133a are connected to each other by wiring. A common ground potential is supplied to the counter electrodes 133a. Note that the counter electrode 133a may not be separated to correspond to the sensing electrodes 131a. That is, the counter electrodes 133a may be formed integrally to correspond to the plurality of sensing electrodes 131a. The counter electrode 133a may be formed of conductive tape, instead of the conductive fabric. For example, the counter electrode 133a may be formed of adhesive copper tape.

The front cover layer 135 is placed on the front surface of the first layer 131. The back cover layer 136 is placed on the back surface of the third layer 133. The front cover layer 135 and the back cover layer 136 may constitute a case containing the first layer 131, the second layer 132, and the third layer 133. For example, the first layer 131, the second layer 132, and the third layer 133 are accommodated between the front cover layer 135 and the back cover layer 136. The front cover layer 135 and the back cover layer 136 are, for example, PVC (polyvinyl chloride) sheets having a thickness of 0.05 mm to 0.5 mm.

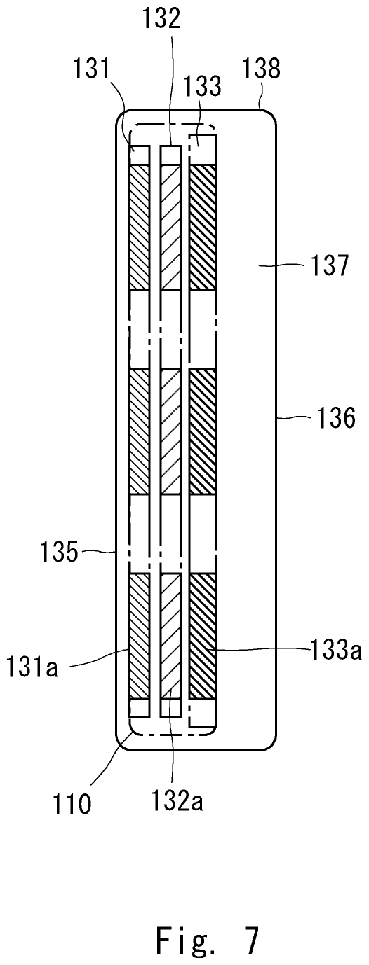

FIG. 7 is a cross-sectional view showing an implementation example of the pressure sensor unit 110. The first layer 131 to the third layer 133 are the same as those in FIG. 6. A cushion layer 137 is placed on the back side of the third layer 133. A foam material such as urethane may be used as the cushion layer 137. This makes the chair more comfortable to sit. The first layer 131, the second layer 132, the third layer 133, and the cushion layer 137 are accommodated in a case 138. The case 138 corresponds to the front cover layer 135 and the back cover layer 136 of FIG. 6.

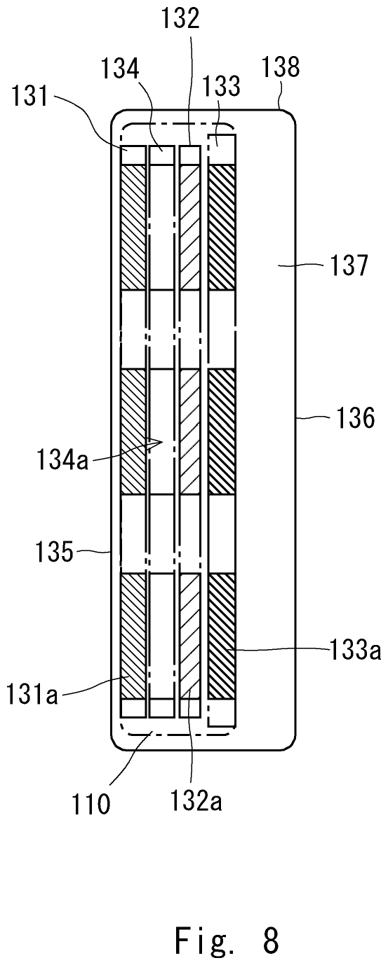

FIG. 8 is a cross-sectional view showing another implementation example of the pressure sensor unit 110. In FIG. 8, a fourth layer 134 is added to the configuration of FIG. 7. The fourth layer 134 is arranged between the first layer 131 and the second layer 132. The fourth layer 134 is formed of a foam material. For example, urethane foam may be used as the foam material of the fourth layer 134. The fourth layer 134 includes openings 134a corresponding to the sensing electrodes 131a. The fourth layer 134 includes nine openings 134a so as to form the nine sensors 111 to 119. Each of the openings 134a has the same size as that of the sensing electrode 131a and overlaps the sensing electrode 131a. The sensing electrode 131a and the conductive sheet 132a are placed to face each other through the opening 134a.

When the pressure received by each of the sensors 111 to 119 exceeds a predetermined value, the first layer 131 and the second layer are brought into contact with each other through the opening 134a. For example, when the sensor 111 receives a certain pressure or more, the sensing electrode 131a corresponding to the sensor 111 is brought into contact with the conductive sheet 132a through the opening 134a.

Although the opening 134a, the sensing electrode 131a, and the counter electrode 133a have the same size, they may have sizes different from each other. The opening 134a, the sensing electrode 131a, and the counter electrode 133a may be placed in such a way that at least a part of them overlaps each other. For example, the opening 134a may be smaller than the sensing electrode 131a. The fourth layer 134 may not be placed between the second layer 132 and the third layer 133 and instead may be placed between the second layer 132 and the third layer 133. In this case, when the sensor 111 receives a certain pressure or more, the counter electrode 133a corresponding to the sensor 111 is brought into contact with the conductive sheet 132a through the opening 134a.

That is, the pressure sensor unit 110 may include the third layer 133, the second layer 132, the fourth layer 134, and the first layer 131 layered in this order from the back side to the front side of the pressure sensor unit 110 or may include the third layer 133, the fourth layer 134, the second layer 132, and the first layer 131 layered in this order from the back side to the front side of the pressure sensor unit 110.

Each of the sensors 111 to 119 detects a pressure according to a change in capacitance generated between the sensing electrode 131a and the counter electrode 133a. Thus, the pressure sensor unit 110 outputs nine pieces of detection data in real time.

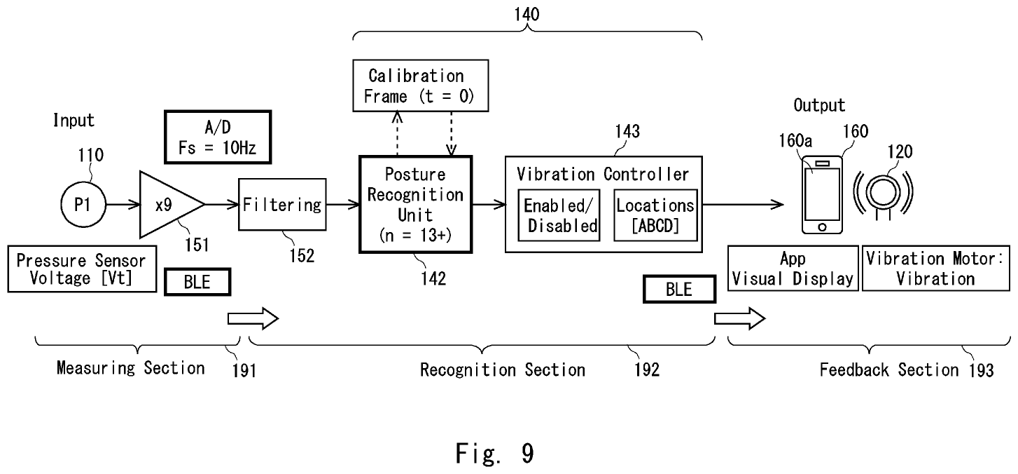

FIG. 9 is a block diagram showing a control system of the posture detection system 1. The posture detection system 1 is broadly divided into a measurement section 191, a recognition section 192, and a feedback section. The posture detection system 1 may be controlled by a software wafer such as a program, hardware such as a circuit, or a combination of them.

The measurement section 191 includes the pressure sensor unit 110 and an A/D converter 151. As described above, the pressure sensor unit 110 includes the nine sensors 111 to 119. Each of the nine sensors 111 to 119 detects a pressure applied from the user's back. Each of the sensors 111 to 119 outputs a detected voltage corresponding to the detected pressure to the A/D converter 151. The A/D converter 151 converts the detected voltage from analog to digital. Then, the detected voltage, i.e., detected pressure, becomes digital detection data. Note that a sampling frequency Fs of the A/D converter 151 is 10 Hz.

The recognition section 192 includes a filter 152, a posture recognition unit 142, and a vibration controller 143. The posture recognition unit 142 and the vibration controller 143 are also referred to as a classification unit 140. A part or all of the processing of the recognition section 192 may be performed by a computer program of the control module 102.

The filter 152 is, for example, a band pass filter. The filter 152 filters a digital signal from the A/D converter 151 and outputs the filtered signal to the posture recognition unit 142.

A digital signal from the filter 152 is input to the posture recognition unit 142 as the detection data. The posture recognition unit 142 recognizes the user's posture based on the detection data. To be more specific, the posture recognition unit 142 can classify the user's postures into 13 or more. Further, the detected pressure in a calibration frame (t=0) is input to the posture recognition unit 142 as reference data. The processing of the posture recognition unit 142 will be described later.

The posture recognition unit 142 outputs a result of the processing to the vibration controller 143. The vibration controller 143 determines whether to cause the vibrators to vibrate based on a result of the classification. The vibration controller 143 determines a vibrator that vibrates and a vibrator that does not vibrate according to the result of the classification. Thus, the vibrator that vibrates changes according to the user's posture. For example, when the user's posture is becoming poor, the vibrator vibrates. This can encourage the user to correct his/her posture.

The feedback section 193 includes a user terminal 160 and the feedback mechanism 120. The feedback mechanism 120 includes the vibrators 121 to 124 as shown in FIG. 4 or vibrators 221, 222, 241 and 242 as shown in FIG. 5. The user terminal 160 is a smartphone, a tablet computer or a PC, and includes a monitor, an input device, a CPU, a memory, a speaker, and so on. The user terminal 160 stores an application program (app) for the posture detection system.

The user terminal 160 includes a display unit 160a that performs a display according to the result of the classification. This enables visual feedback to be provided to the user. The vibrators 121 to 124 operate in accordance with a control signal from the vibration controller 143. By doing so, feedback can be provided to the user. Further, the vibrators 221, 222, 241, and 242 of the seating face sensor unit 201 may operate in accordance with a control signal. In this way, the vibrators 121 to 124 and the vibrators 221, 222, 241, and 242 vibrate according to the result of posture classification.

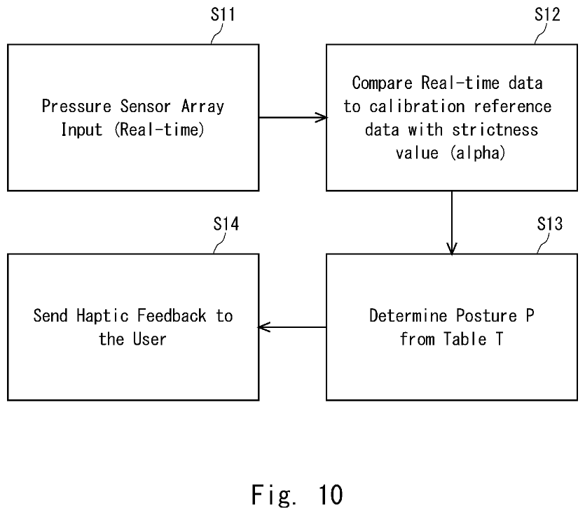

FIG. 10 is a flowchart of a posture detection method carried out by the posture detection system. Firstly, a detected pressure detected by the pressure sensor unit 110 is input to the classification unit 140 (S11). The pressure sensor unit 110 detects a pressure in real time. That is, the pressure sensor unit 110 outputs the latest detected pressure to the classification unit 140 as needed. The latest detected pressure is referred to as real-time data.

Next, the posture recognition unit 142 compares the real-time data with the reference data using a threshold α (S12). The reference data is detection data in a calibration frame (t=0). For example, the calibration can be done at the time t=0 when the user sits on the chair 2. When the user sits on the chair 2, the user terminal 160 outputs a message for encouraging the user to sit with a good posture (upright posture). Then, the pressure sensor unit 110 and the seating face sensor unit 201 detect pressures while the user is sitting with a good posture. This detected pressures are defined as the reference data.

The posture recognition unit 142 calculates a difference value εi between the real-time data and the reference data. Next, the posture recognition unit 142 compares the difference value εi with the threshold α. The difference value εi is calculated by the following formula (1), where Vt is the real-time data, and Vo is the reference data.

εi=(Vt−Vo)² (1)

The difference value εi indicates a difference between the pressure applied when the posture is correct and the pressure with the current posture, because the reference data Vo is the pressure applied when the user sits with a correct posture. The posture recognition unit 142 determines whether the difference value εi exceeds the threshold α. When the difference value εi exceeds the threshold α, a deviation from the pressures applied when the posture is correct is large. When the difference value εi is less than or equal to the threshold α, the pressure is close to the pressure applied when the posture is correct.

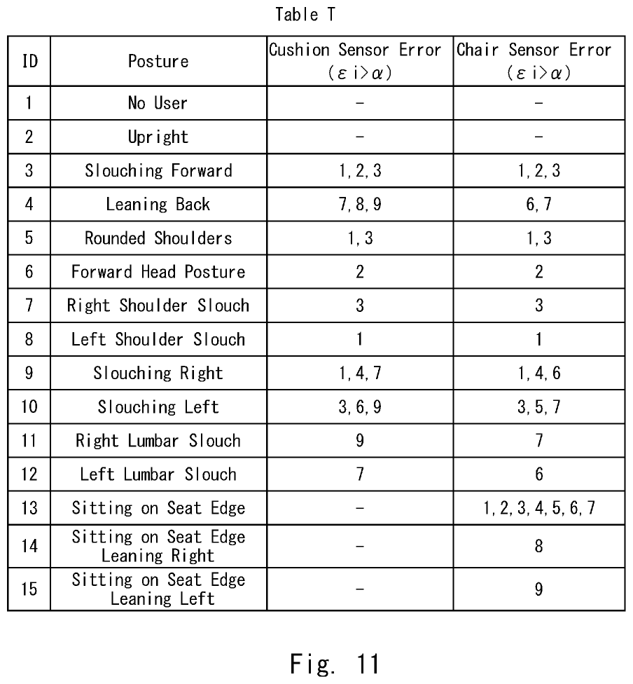

Next, the posture recognition unit 142 determines a posutre P with reference to the table T (S13). An example of the table T is shown in FIG. 11. In the table T shown in FIG. 11, the posutres P are classified into 15 postures. For each posture, the position of the sensor having the difference value εi exceeding the threshold α is shown. The positions of the sensors 111 to 119 in the pressure sensor unit 110 are indicated by the positions 1 to 9 in FIG. 4. The positions of the sensors 211 to 219 in the seating face sensor unit 201 are indicated by the positions 1 to 9 in FIG. 5.

For example, with ID=3, the difference value εi exceeds the threshold for the sensors 111 to 113 at the positions 1 to 3 of the pressure sensor unit 110. Furthermore, the difference value εi exceeds the threshold for the sensors 211 to 213 at the positions 1 to 3 of the seating face sensor unit 201. Thus, the user's posture P is classified as “Slouching forward”.

The vibrators 121 to 124, 221, 222, 241, and 242 output haptic feedback to the user (S14). That is, the vibration controller 143 outputs control signals corresponding to a result of the classification to the vibrators 121 to 124, 221, 222, 241, and 242. Then, haptic feedback can be provided according to the classified posture P.

The posture detection system 1 may provide visual feedback or audial feedback in combination with the haptic feedback. For example, the user terminal 160 may display a message or the like on the display unit according to the result of the classification. Alternatively, the user terminal 160 may output a message from a speaker according to the result of the classification.

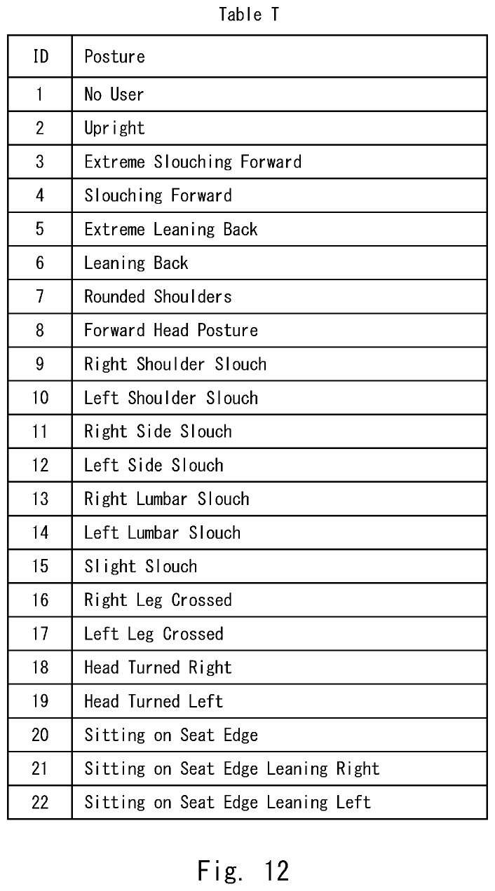

Further, the table T shown in FIG. 11 is an example of this embodiment, and the number of classifications and the classified postures are not limited to those in the table T of FIG. 10. For example, the table T shown in FIG. 12 may be used. In FIG. 11, the postures are classified into 22 postures.

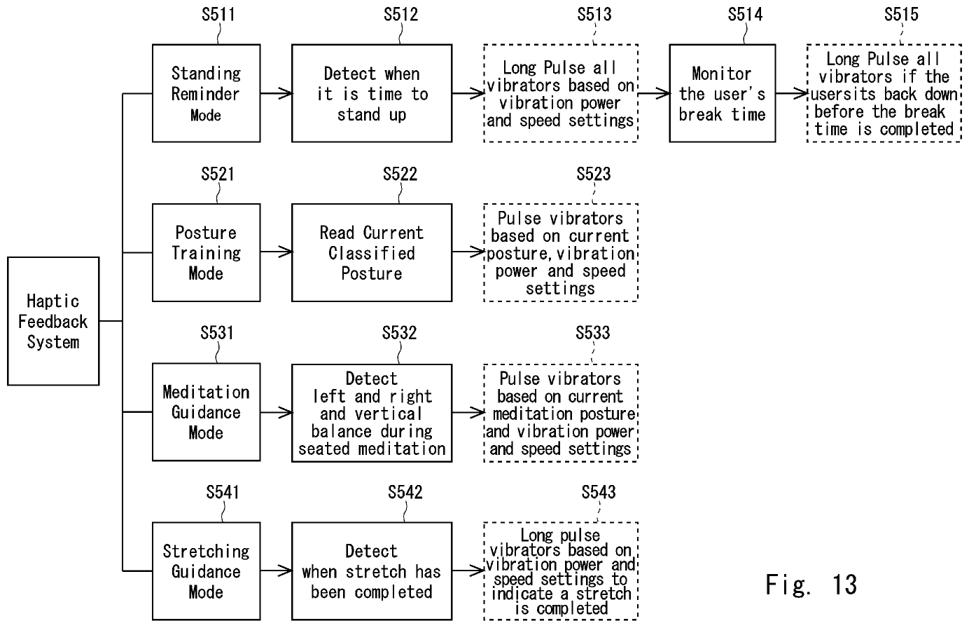

FIG. 13 is a drawing showing an example of the haptic feedback. FIG. 13 shows a flow for providing the haptic feedback in four modes. The user can select each mode. As a matter of course, the user may select one mode or two or more modes at the same time. In each mode, the power and speed for operating the vibrators are set in advance.

When a standing reminder mode is selected (S511), the time for the user to stand up is detected (S512). All vibrators are operated with long pulses at the set power and speed (S513). For example, when the user is sitting continuously for a certain period of time or longer, the posture detection system 1 can output a standing reminder using vibrators.

Then, the posture recognition unit 142 monitors the user's break time (S514).

When the user is seated before the user's break time is over, the vibration controller 143 operates all the vibrators with long pulses (S515). That is, when the user is seated before the break time reaches a preset time, the break is insufficient. Thus, the vibration controller 143 controls the vibrators to output a standing reminder again. The user can take breaks for an appropriate period of time at an appropriate interval.

When a posture training mode is selected (S521), the posture recognition unit 142 reads the classified current posture (S522). The vibration controller 143 controls the vibrators to be pulsed according to the current posture (S523).

When a meditation guidance mode is selected (S531), the posture recognition unit 142 detects the left/right balance and the vertical balance during meditation (S532). The vibration controller 143 controls the vibrators to be pulsed according to the current posture (S533).

When a stretch guidance mode is selected (S541), the posture recognition unit 142 detects that the stretch has been completed (S543). In order to indicate that the stretch has been completed, the vibration controller 143 controls the vibrators to operate with long pulses (S543).

In the posture training mode, meditation guidance mode, and stretch guidance mode, the posture to be taken by the user is presented. For example, the display unit 160a can display an image of a pose such as a training pose, a meditation pose, or a stretch pose, thereby encouraging the user to change his/her posture. The posture to be presented may be shown by an image or a message.

The pressure sensor unit 110 or the seating face sensor unit 201 detects the pressures applied from the user. The user terminal 160 can determine whether the user's current posture matches the presented posture. The display unit 160a displays a recommended pose. The user terminal 160 determines whether the user's pose matches the recommended pose according to a result of the detection of the pressure sensor unit 110, and provides feedback according to a result of the determination.

For example, a template is prepared for each pose to be presented. That is, the control module 102 or the user terminal 160 stores, for example, a pressure distribution serving as a template in a memory or the like. By comparing the pressure distribution of the template in the user terminal 160 with the current pressure distribution, it is possible to determine whether the user's pose is the same as the recommended pose. The template may be a pressure distribution measured in advance for each user. Alternatively, a template measured for a certain user may be applied to another user. In this case, the template may be calibrated according to the user's physical information such as the user's height, weight, body mass index, etc. That is, the pressure distribution of the template may be corrected according to the user's physical information.

(Vitals Sensor)

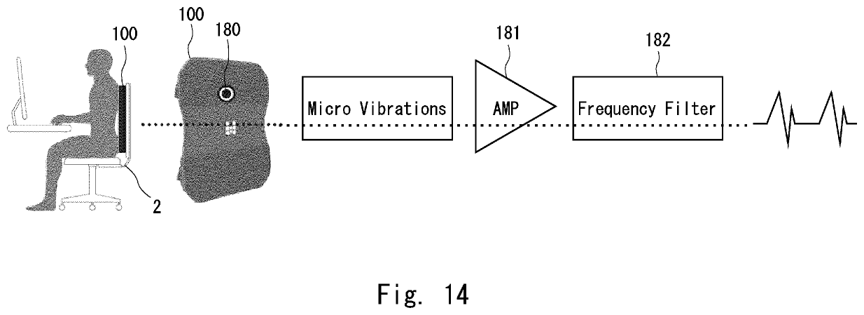

The backrest cushion 100 may include a vibration sensor that can detect the user's vital information. FIG. 14 is a drawing for describing detection of vital information carried out by a vibration sensor 180. The vibration sensor 180 is a piezo element or a microphone, and measures vibrations applied from the user. A measurement signal from the vibration sensor 180 is amplified by an amplifier 181. Then, the amplifier 181 outputs the amplified measurement signal to a frequency filter 182. The frequency filter 182 passes a signal in a predetermined frequency band. The amplifier 181 and the frequency filter 182 are mounted on, for example, the control module 102. The vital information is a respiration rate or a heart rate (HR).

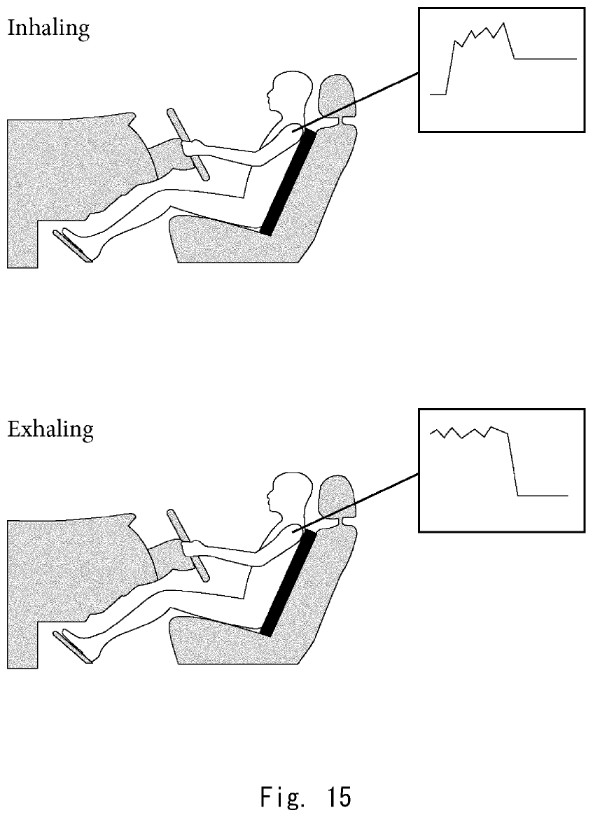

FIG. 15 shows an example in which the respiration rate is measured using the vibration sensor 180. Waveforms when a person inhales differ from waveforms when the person exhales. Thus, the control module 102 can calculate the respiration rate from periods of the waveforms of the vibration sensors. Alternatively, the heart rate may be acquired.

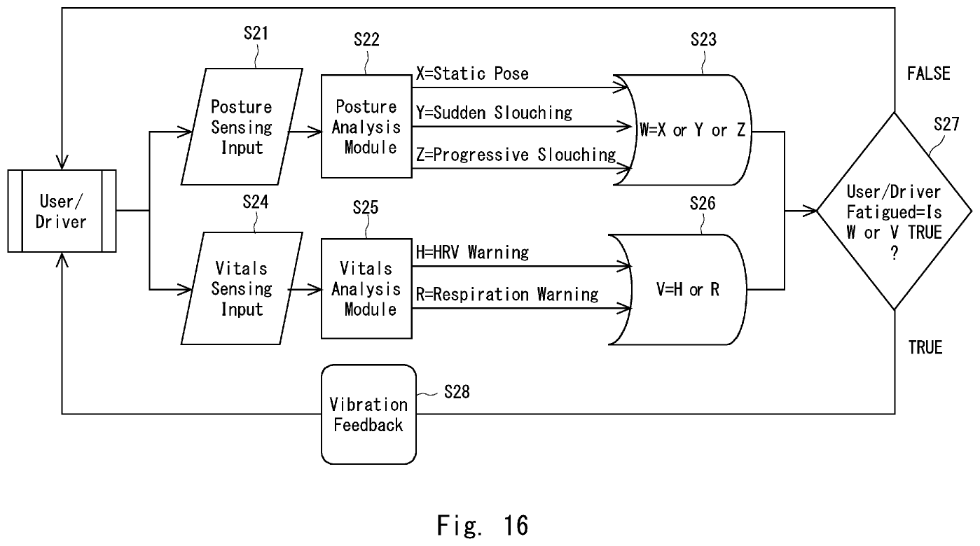

Next, a method for estimating the user's fatigue level using vital information will be described. FIG. 16 is a flowchart for describing processing for estimating the fatigue level. The posture detection system 1 determines whether the user is fatigued or not.

When the user sits on the chair, the posture detection system 1 senses his/her posture (S21). That is, a detection signal corresponding to the pressure applied to the pressure sensor unit 110 or the like is input to the control module 102. Next, a posture analysis module of the control module 102 determines whether the posture corresponds to any of (X) static pose, (Y) sudden slouching, and (Z) progressive slouching (S22). The posture analysis module can make this determination by comparing the latest posture with the previous posture. Then, the control module 102 calculates a logical sum W of (X), (Y), (Z) (S23).

Further, the posture detection system 1 senses the vital information (S24). That is, the vibration received by the vibration sensor 180 from the user is measured. Then, the vital information analysis module of the control module 102 analyzes the vital information (S25). Specifically, the vital information analysis module determines whether (H) the heart rate is at a warning level and (R) whether the respiration rate is at a warning level. For example, the vital information analysis module conducts an analysis by comparing the measured heart rate and respiration rate with the respective thresholds. Next, the vital information analysis module calculates a logical sum (V) of (H) and (R) (S26).

In parallel with S21 to S23, when one of W and V is true, the control module 102 determines that the user is fatigued. That is, when any one of (X), (Y), (Z), (H), and (R) is applicable, it is assumed that the user is fatigued. When it is determined that the user is fatigued (YES in S27), a feedback mechanism provides vibration feedback. In other words, the vibrators 121 to 124 vibrate. When it is determined that the user is not fatigued (NO in S27), the feedback mechanism does not provide vibration feedback. The above processing is repeated.

In this manner, the user's fatigue level can be estimated. That is, when the user is fatigued, the posture detection system 1 provides feedback to encourage the user to take a break. In the above description, the posture detection system 1 determines whether the user is fatigued. Alternatively, a fatigue score may be calculated in order to estimate the fatigue level based on the classified postures.

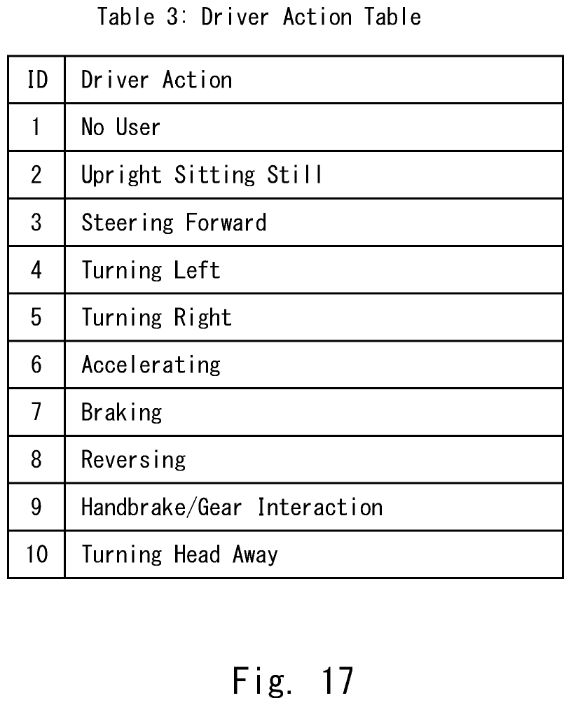

Furthermore, the pressure sensor unit 110 may be mounted on a driver's seat of a vehicle. Note that the pressure sensor unit 110 may be detachable from the driver's seat, or may be built into the driver's seat in advance. The actions of the user who is a driver can also be classified using the pressure sensor unit 110. FIG. 17 is a table in which driving actions are classified. A pressure distribution template is prepared for each action. In FIG. 17, the user's driving actions are classified into eight actions. Actions other than the driver action may be used for the estimation, as a matter of course.

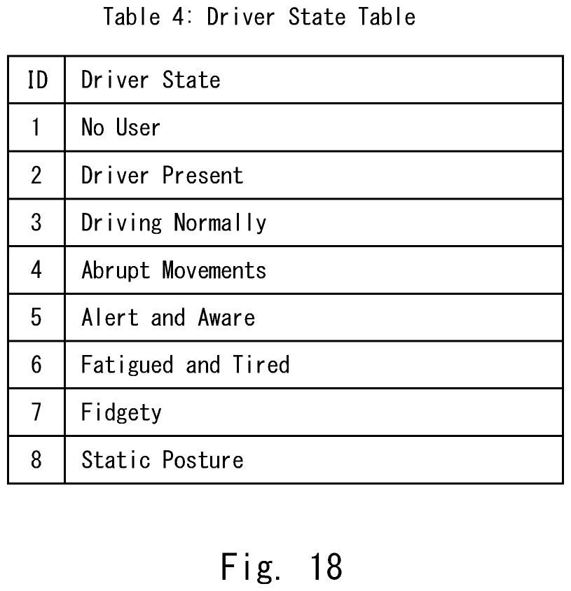

Furthermore, the user's states can be classified according to a result of classifying an operation. FIG. 18 shows a table in which user states are classified. For example, when there are many abrupt movoments or when there is no change in the user's movement for a certain period of time, the user may be fatigued. Thus, the user's state can be predicted according to a time in which the classified action lasts, an interval of action changes, a percentage of the action, etc. Thus, the user's state can be predicted according to a result of the action classification. In this case, the vital information such as the user's heart rate may be used together with the above-listed items. The user terminal may predict the action and state from the pressure distribution. A machine learning model may be used for such classification of actions or states.

(Reminder)

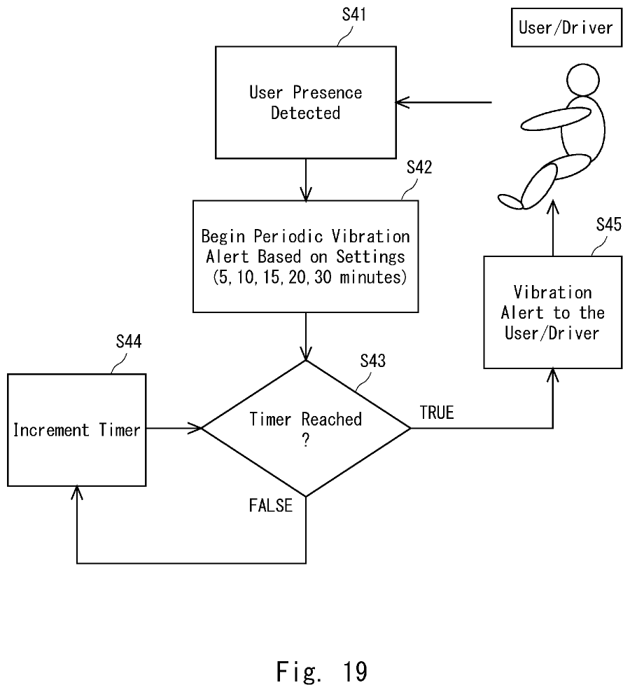

FIG. 19 is a flowchart showing processing for outputting a periodic reminder to the user. Here, the feedback mechanism 120 outputs a vibration alert to encourage the user such as a driver to take a periodic break. The vibration alert may function as a standing reminder. As a matter of course, visual feedback may be provided by a display monitor or audial feedback may be provided by a speaker.

First, the pressure sensor unit 110 or the seating face sensor unit 201 detects the presence of the user (S41). For example, the control module 102 recognizes that the user is sitting on the chair 2 when the detected pressure of one or more sensors becomes a predetermined value or more. Next, the control module 102 begins a periodic vibration alert timer based on a set time (S42). Any time may be set as the set time. For example, the set time may be, 5, 10, 15, 20, or 30 minutes. The user may change the set time to any value, as a matter of course.

Next, the control module 102 determines whether the timer has reached the set time (S43). When the timer has not reached the set time (FALSE in S43), the control module 102 increments the timer (S44) and performs the determination in S43 again. When the timer has reached the set time (TRUE in S43), the feedback mechanism 120 outputs a vibration alert.

In this manner, a reminder or an alert can be output to the user periodically. This encourages the user to take a break at an appropriate timing.

(Stretch Guidance Mode)

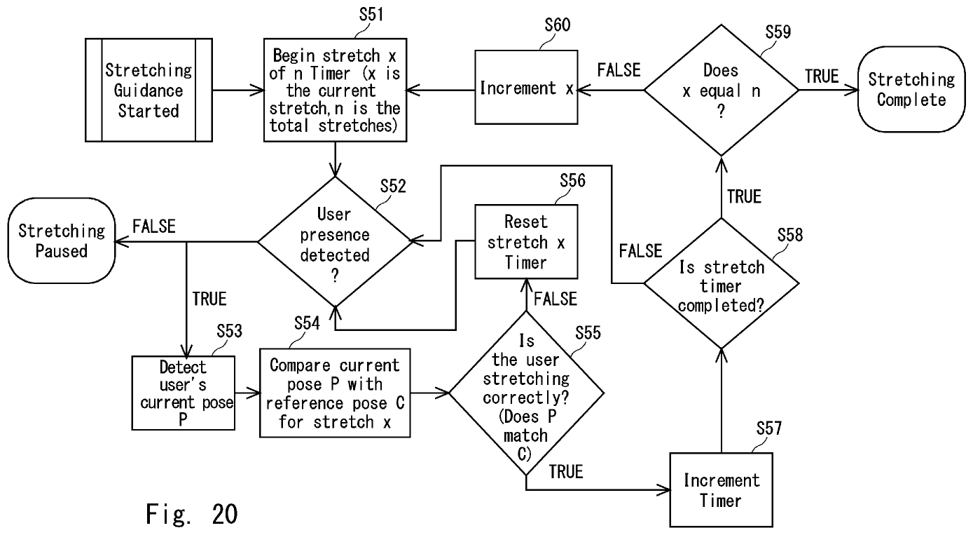

FIG. 20 is a flowchart for processing in the stretching guidance mode. Here, an example in which n stretch poses (n is an integer of 1 or greater) are presented to the user is shown. The current stretch number is defined as x (x is an integer of 1 to n). Further, a stretch pose to be taken by the user is defined as a reference pose C. Thus, the user stretches by posing as the first to nth reference poses.

When the stretch guidance mode is selected, a timer for stretch x of n is begun (S51). Next, the pressure sensor unit 110 and the seating face sensor unit 201 detect whether the user is present (S52). When the user is not present (FALSE in S52), the stretching is paused. When the user is present (TRUE in S52), the pressure sensor unit 110 or the like detects the user's current pose P (S53). The display unit 160a displays an image of the reference pose C as a recommended pose. The user watches the image of the reference pose C and takes the stretch pose. Then, the control module 102 compares the current pose P with the reference pose C of the stretch x (S54).

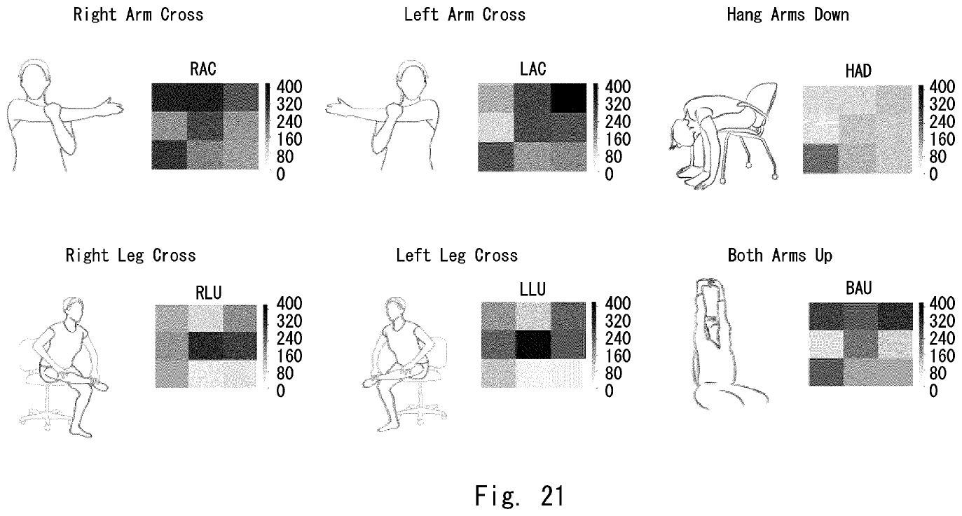

FIG. 21 is a drawing schematically showing pressure distributions for six stretch poses. Specifically, stretch poses of right arm cross, left arm cross, hang arms down, right leg cross, right leg cross, left leg cross, and both arms up are shown in the drawing. Further, typical pressure distributions of the sensors 111 to 119 in the respective stretch poses are shown as templates in the drawing. The user may stretch with poses other than the stretch poses shown in FIG. 21, as a matter of course. The template is preferably measured for each user. It is needless to say that a single template of the user may be used for another user.

The control module 102 determines whether the user is correctly stretching (S55). The control module 102 determines whether the current pose P matches the reference pose C. For example, when the reference pose C is right arm cross, the control module 102 determines whether the current pressure distribution matches the pressure distribution of the right arm cross shown in FIG. 21. Obviously, the pressure distribution of the current distribution does not need to completely match the pressure distribution of the reference pose C. That is, the control module 102 may compare the pressure distributions with some tolerance.

When the pose P does not match the reference pose C (FALSE in S55), the stretch x timer is reset (S56), and the process returns to Step S52. At this time, the display unit 160a may display a message or the like in order to notify the user that the current pose P is not a correct reference pose.

When the current pose P matches the reference pose C (TRUE in S55), the control module 102 increments the timer (S57). Then, the control module 102 determines whether the stretch x timer has completed (S58). When the timer has not completed (FALSE in S58), the process returns to S52. In S58, it is determined whether the user has properly stretched for a certain period of time or longer.

When the timer has completed (TRUE in S58), the control module 102 determines whether the number of stretches x is equal to n. When the number of stretches x is not equal to n (FALSE in S59), x is incremented (S60). Then, the process returns to S51, and the above-described processing is performed. When the number of stretches x becomes equal to n (TRUE in S59), the processing ends.

In this way, the user can go through a predetermined number of stretch poses. Furthermore, the user stretches with each stretch pose for a preset time or longer. By doing so, the user can stretch effectively. In S58, when the stretch timer is completed, visual feedback or haptic feedback may be provided to the user so that the user shifts to the next stretch pose. In this way, the display unit 160a displays the stretch poses as the recommended poses. It is determined as to whether the user's pose matches the recommended pose according to a result of the detection by the pressure rensitive sensor unit 110, and feedback is provided according to a result of the determination.

(Meditation Guidance Mode)

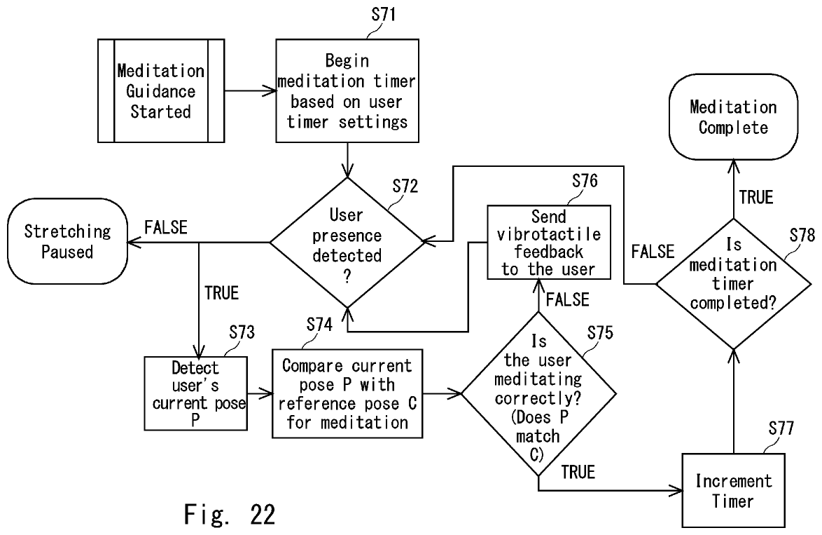

FIG. 22 is a flowchart showing processing in the meditation guidance mode. In the control module 102, a typical meditation pose is registered as the reference pose C. With the meditation pose, the user is balanced in the left/right and vertical directions.

When the meditation guidance mode is selected, the meditation timer is begun (S71). Next, the pressure sensor unit 110 and the seating face sensor unit 201 detect whether the user is present (S72). When the user is not present (S72 FALSE), the meditation is paused. When the user is present (TRUE in S72), the pressure sensor unit 110 or the like detects the user's current pose P (S573). The display unit 160a displays an image of the meditation pose as a refference pose C. The user watches the image of the reference pose C and takes the meditation pose. Then, the control module 102 compares the current pose P with the reference pose C for meditation (S74). That is, by comparing the pressure distribution of the current pose P with the pressure distribution of the reference pose C, it is possible to determine whether the user is posing with an appropriate meditation pose.

The control module 102 determines whether the user is posing with a correct meditation pose (S75). The control module 102 determines whether the current pose P matches the reference pose C. Obviously, the pressure distribution of the current distribution does not need to completely match the pressure distribution of the reference pose C. That is, the control module 102 may compare the pressure distributions with some tolerance.

When the current pose P does not match the reference pose C (FALSE in S75), the feedback mechanism 120 outputs vibrotactile feedback to the user (S76). Then, it can be recognized that the user is not posing as a correct meditation pose. Next, the process returns to Step S72, and the above-described processing is performed. Note that visual feedback may be provided instead of vibrotactile feedback. Alternatively, visual feedback may be provided together with vibrotactile feedback.

When the pose P matches the reference pose C (TRUE in S75), the control module 102 increments the timer (S77). Then, the control module 102 determines whether the stretch x timer has completed (S78). When the timer has not completed (FALSE in S78), the process returns to S72. In S78, it is determined whether the user has medidated with the reference pose C for a certain period of time or longer.

When the timer has completed (TRUE in S78), the meditation is completed. In this manner, the user can pose as a correct meditation pose for a predetermined period of time. As described above, the display unit 160a displays the meditation pose as the recommended pose. It is determined as to whether the user's pose matches the recommended pose according to a result of the detection by the pressure sensitive sensor unit 110, and feedback is provided according to a result of the determination.

(Pain Relief)

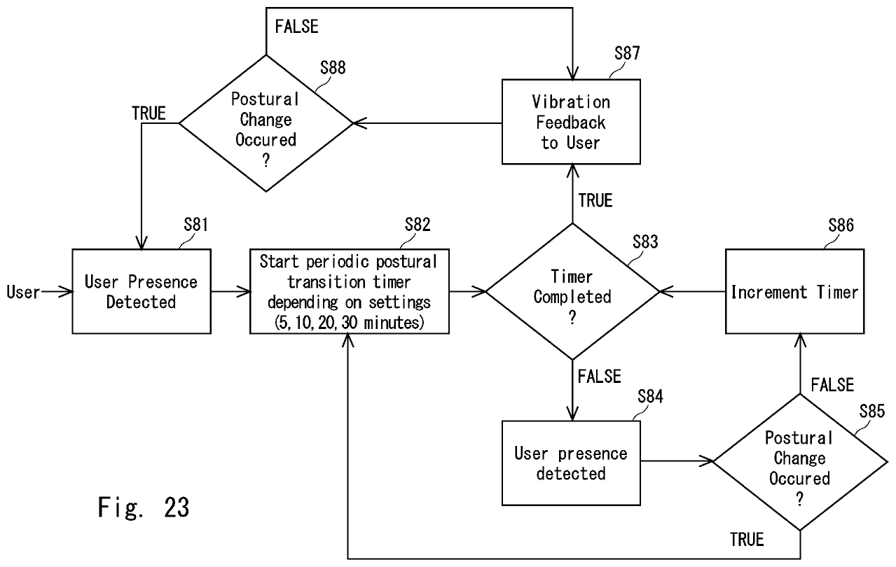

Next, processing for reducing pain for a wheelchair user will be described with reference to FIG. 23. FIG. 23 is a flowchart showing pain reduction processing. FIG. 23 shows processing for reducing pain of the user sitting in the wheelchair. Specifically, when the user has been in the same posture for a certain period of time or longer, feedback is provided to encourage the user to change his/her posture. Specifically, since pain occurs when the user continues to pose with same posture for a certain period of time or longer, the posture detection system 1 performs feedback processing for reducing the pain.

Firstly, when the pressure sensor unit 110 and the seating face sensor unit 201 detect the user (S81), the control module 102 starts a periodic postural transition timer based on a set time (S82). Any time may be set as the set time. For example, the set time may be, 5, 10, 20, or 30 minutes. The user may change the set time to any value, as a matter of course.

Next, the control module 102 determines whether the timer has reached the set time (S83). When the timer has not reached (FALSE in S83), the presence of the user is detected (S84). Then, the control module 102 determines whether the user's posture has changed (S85). When the postural change occurs (TRUE in S85), the process returns to S82, and the timer is started again. When the user's posture has not changed (FALSE in S85), the timer is incremented (S86). Then, the process returns to S83, and the process is repeated until the timer reaches the set time. In S83, it is determined whether the user has not changed his/her posture for a certain period of time.

When the timer reaches the set time (TRUE in S83), the feedback mechanism 120 outputs vibration feedback to the user (S87). That is, when the user has not changed his/her posture for the set time or longer, the feedback mechanism 120 provides vibration feedback to encourange the user to change his/her posture. Next, the control module 102 determines whether the user has changed his/her posture (S88). When the user has changed his/her posture (TRUE in S88), the process returns to S81. When the user has not changed his/her posture (FALSE in S88), the process returns to S87 to provide vibration feedback. By doing so, vibration feedback is continuously output until the user changes his/her posture. Thus, it is possible to encourage the user to change his/her posture and to reduce pain.

(Exercise Member)

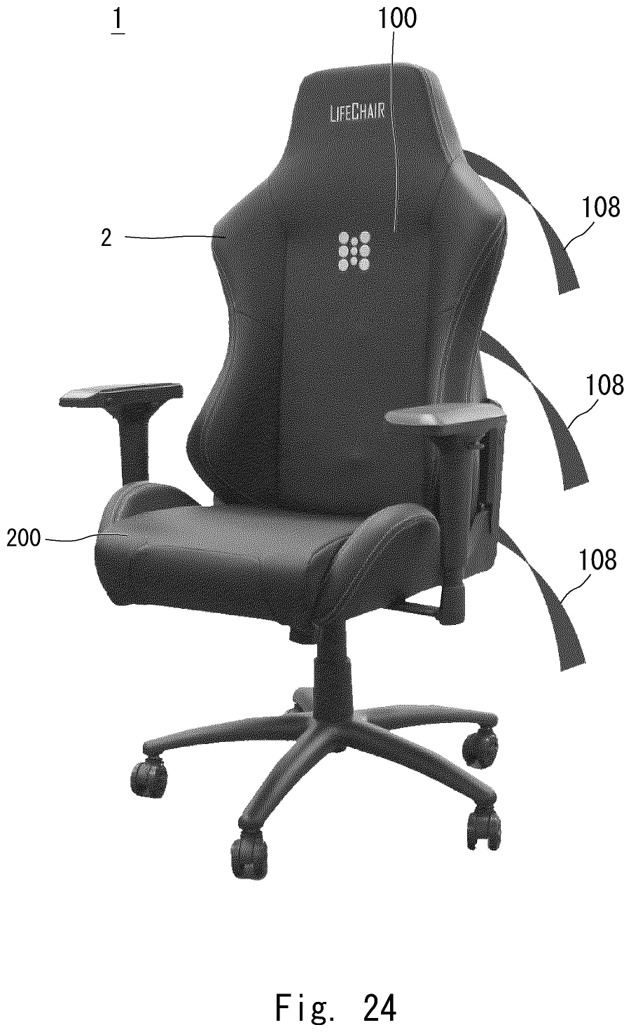

FIG. 24 is a drawing showing a posture detection system 1 according to a modified example. The posture detection system 1 is built into the chair 2. Further, elastic bands 108 are provided on the back side of the chair 2. Each of the elastic bands 108 functions as an exercise member used by the user. The user can exercise using the elastic bands 108. That is, the user performs exercise by grasping and pulling the elastic bands 108, and the pressure sensor unit 110 and the seating face sensor unit 201 can also detect the posture during exercise. Obviously, an extendable tube or the like may be used as the exercise member instead of the elastic band 108.

(Health Care Report)

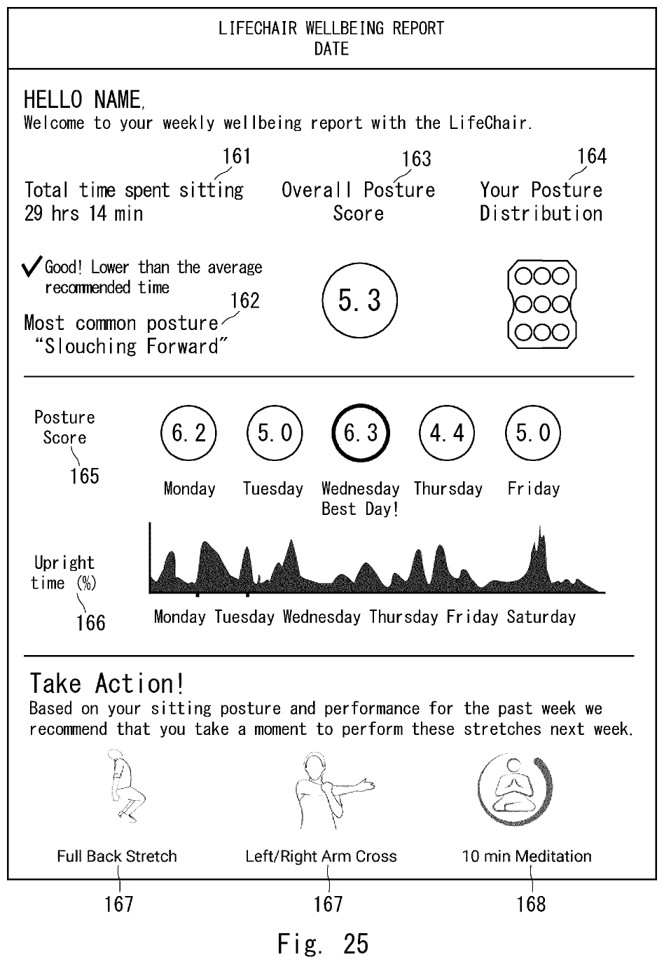

The posture detection system 1 can also display a health care report by analyzing the user's posture. FIG. 25 is a display screen showing an example of a health care report displayed on the user terminal 160. The user terminal 160 can analyze the user's posture and create a report periodically. An interval at which a report is created may be, for example, daily, weekly, monthly, etc. That is, the display unit 160a can display daily reports, weekly reports, and monthly reports on the user's postures. FIG. 25 shows a report summarizing the posture for one week.

The report includes a sittng time 161, a most common posture 162, a posture score 163, a posture distribution (pressure distribution) 164, and so on. The posture score is a value obtained by evaluating the user's posture in 10 levels, where 10 is the highest posture score, while 1 is the lowest posture score. The report displays the posture score 165 for each day between Monday to Friday. Here, the posture score of Wednesday is highlighted, because it is the highest. A percentage 166 of the upright posture every hour is also shown. The longer the upright posture, the higher the posture score becomes.

The report also shows recommended stretch poses 167 and a recommended meditation time 168. The user terminal 160 analyzes the user's posture and suggests a stretch pose 169 suitable for the user. That is, the posture detection system 1 can encourage the user to stretch for correcting the distortion of the user's posture. Additionally, the posture detection system 1 can present meditation at an appropriate time to reduce fatigue.

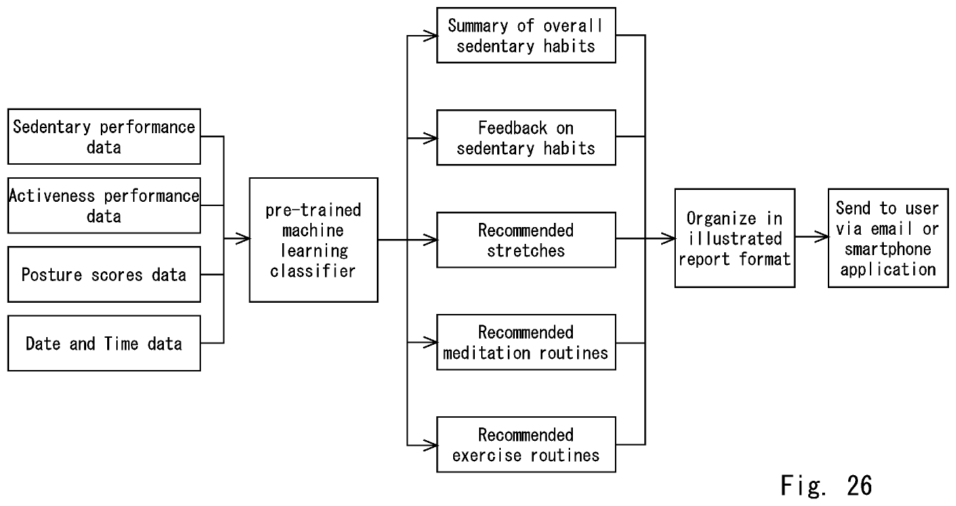

FIG. 26 is a flowchart showing processing for outputting a report. Data of sedentary performance, activeness performance, posture scores, and date and time is input to a machine learning model. The machine learning model generates the following output data (1) to (5) from these pieces of input data.

(1) Summary of overall sedentary habits

(2) Feedback on sedentary habits

(3) Recommended stretches

(4) Recommended meditation routines

(5) Recommended exercise routines

For example, the posture detection system 1 determines amount of time spent sitting down per a certain time period. The certain time period is, for example one day, one week, or one month. The posture recognition unit 142 classifies the posture based on the pressure distribution and stores the data of the classification result in the time period. The posture detection system 1 calculates the percentage of the posture classified by the posture recognition unit 142 For example, the posture detection system 1 calculates the percentage of the upright posture as a correct posture. The posture detection system 1 may determine the most common posture based on the percentage of the posture. The most common posture may be a posture with the highest percentage in the certain time period. The posture detection system 1 may determine frequency of breaks per the time period. The posture detection system 1 may determine performance of stretches or meditation (T/F). As described above, the posture detection system 1 can output the summary of overall sedentary habits including the percentage of the classified posture, frequency of the breaks.

The posture detection system 1 compares values and trends in summary of overall sedentary habits to average values in a given population/group. The posture detection system 1 defines the ideal values such as the percentage of the classified posture, the frequency of the breaks or the like from the average values in the given population/group. The posture detection system 1 compares values and trends in summary of overall sedentary habits to pre-defined ideal values in a given population/group. Therefore, the posture detection system 1 performs the feedback of the sedentary habits to the user.

The posture detection system 1 can calculate the posture score 163 for the certain time period based on at least one of data such as the sitting time duration, the percentage of occurrence of the posture, the frequency of breaks, the duration of breaks, symmetry value of the pressure distribution and a detection of the performance of stretches. The posture detection system 1 may calculate the symmetry value of the pressure distribution detected by the pressure sensor unit.

The posture detection system 1 can recommend action for improving the posture score 163. The display unit displays the stretching pose, or the meditation routines, the exercise pose, or the like. The user takes the stretch pose, the meditation routines or the exercise routines for improving the posture score 163. The posture detection system can recommend the predefined stretches poses. The stretches pose is associated with the user posture classified by the classifier. That is, a pair of the user's postures and stretch poses are stored in memory or the like. The posture detection system can recommend the meditation routines or the exercise routines in a way similar to the method in recommending stretches, but can recommend consecutive balance shifts instead of predefined stretch poses.

The display unit displays an image indicating information of a stretching pose for guiding the user to perform stretches when a stretch guidance mode is selected. The posture detection system 1 may determine whether a current pose of the user matches the stretching pose based on a ranking of a similarity metric between the stretch pose pressure distribution and the posture pressure distribution. The posture detection system 1 may determine at least the cosine similarity between the stretch's pressure distribution and the user's historic posture pressure distribution. The posture detection system 1 may rank the stretch poses according to at least a value of the cosine similarity between the stretches pressure distributions and the user's historic postures pressure distribution. The posture detection system 1 may pair the user's historic posture with its least similar stretch pose.

The posture detection system 1 can include a machine learning tool (algorithm) that can output the sedentary guidance suggesting the exercise routines, the meditation routines, poses or the like. The sedentary guidance may be information suggesting the break schedule and recommendation for standing remainder and seating regulation. The machine learning tool may be a supervised machine leaning tool, an unsupervised machine learning tool, or the like. In this embodiment, the machine learning tool is the supervised machine learning tool. The input data of the supervised machine learning classifier may include a history of the user's postures and a score of the posture or activeness of the user. The output data of the supervised machine learning classifier suggests the pose based on the input data. the stretch pose is associated with the classified posture and wherein the sedentary guidance is classified based on a history of the user's postures and a score of the posture or activeness of the user.

The posture detection system 1 can include another supervised machine learning tool (algorithm) that output the user posture based on the pressure distribution. This supervised machine learning tool may classify the user posture with using random forest, k-nearest neighbors, a neural network, etc. or their combination. The input data of the supervised machine learning tool includes information of the physical features of the user such a body mass index value and the detection data of the pressure sensor unit.

The posture detection system 1 can include another supervised machine learning tool (algorithm) that output a behavior or action of user other than the posture of the user. This supervised machine learning tool may estimate the behavior or action of the user based on the pressure distribution. This supervised machine learning tool may use random forest, k-nearest neighbors, a neural network, etc. or their combination. The input data of the supervised machine learning tool includes user's physical features information such a body mass index value, the user's vital information, the detection data of the pressure sensor unit, a score of the posture or activeness, and the time of the day.

At least a part of the process as mentioned in the embodiment may be executed by one or more remote server or the like. The supervised machine learning tool can be a computer algorithm or processing circuity, or their combinations.

Then, the output data of (1) to (5) are organized into a format shown in FIG. 25. Then, the organized output data is sent to the user via an email or a smartphone application.

(Machine Learning Model)

Hereinafter, an embodiment that uses a machine learning model will be described. Note that a program to be a learned model may be stored in the user terminal 160 or in a network server. When a program to be a learning model is stored in the user terminal 160, it can be incorporated into an application. When a program to be a learned model is stored in the server, the user terminal 160 sends data of the detected pressure and result of the classification to the server using WiFi communication or the like. The server transmits a result of executing the machine learning model to the user terminal 160. The learned model functions as a classifier.

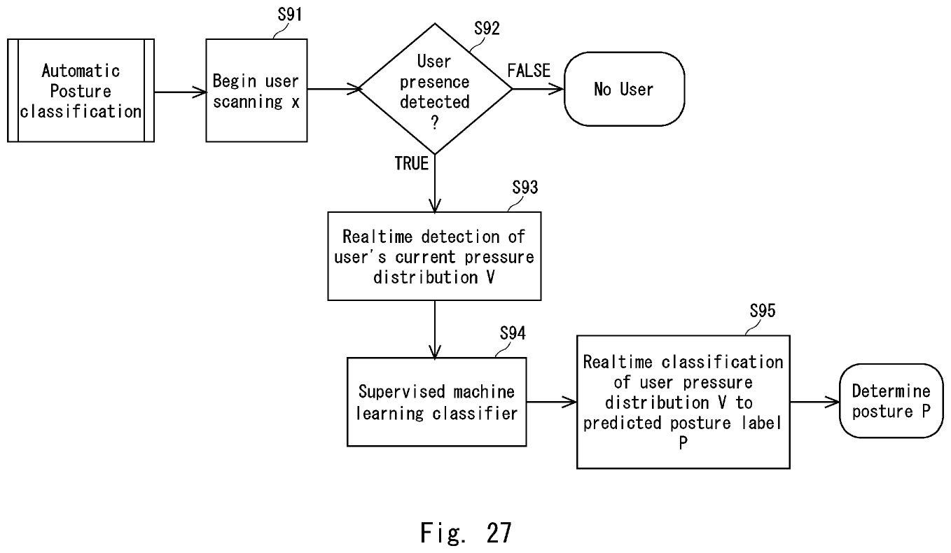

FIG. 27 is a flowchart showing a method for classifying postures using a machine learning model. Here, a machine learning model pre-trained on learning data is used as a classifier. For example, supervised learning is used as the learning method. The pressure distribution data for a user X is acquired in advance as the learning data. Furthermore, the user X's posture when the pressure distribution data is acquired and associated with the learning data as a correct answer label (teacher data).

The pressure distribution data includes detected pressures of the pressure sensor unit 110 and the seating face sensor unit 201. When only the pressure sensor unit 110 is used, the pressure distribution data includes, for example, data of nine detected pressures. When the pressure sensor unit 110 illustrated in FIG. 4 and the seating face sensor unit 201 illustrated in FIG. 5 are used, the pressure distirbution data includes, for example, data of 18 detected pressures. When the pressure sensor unit 110 illustrated in FIG. 4 is used, the pressure distirbution data includes, for example, data of 9 detected pressures. In the learning data, the detected pressure of each sensor is associated with a posture that is a correct answer label.

The classifier is generated by performing supervised machine learning in advance using the learning data including the correct answer label. The program that becomes the classifier performs the following processing.

First, the user X is scanned (S91). That is, the user of the user terminal 160 that has accessed the control module 102 is identified. For example, in the case of a smartphone, a user is identified for each user terminal 160. When the user terminal is, for example, a shared personal computer, the user X may be identified by a login ID, etc.

Then, the presence of the user is detected (S92). For example, it is determined as to whether the user is sitting according to the detected pressure of the sensor. When the presence of the user has not been detected (FALSE in S92), the user is not sitting, and the process ends. When the presence of the user is detected (TRUE in S92), the pressure sensor unit 110 or the like detects a current pressure distribution V of the pressure applied from the user in real time (S93). As described above, the control module 102 acquires the detected pressure from each sensor as the current pressure distribution V.

The pressure distribution V is input to the classifier that has learned by supervised machine learning (S94). The classifier outputs a posture label expected from the pressure distribution V, thereby classifying the user's posture in real time (S95). Then, the pose P is determined. In this manner, the user's postures can be classified as appropriate by using the machine learning model.

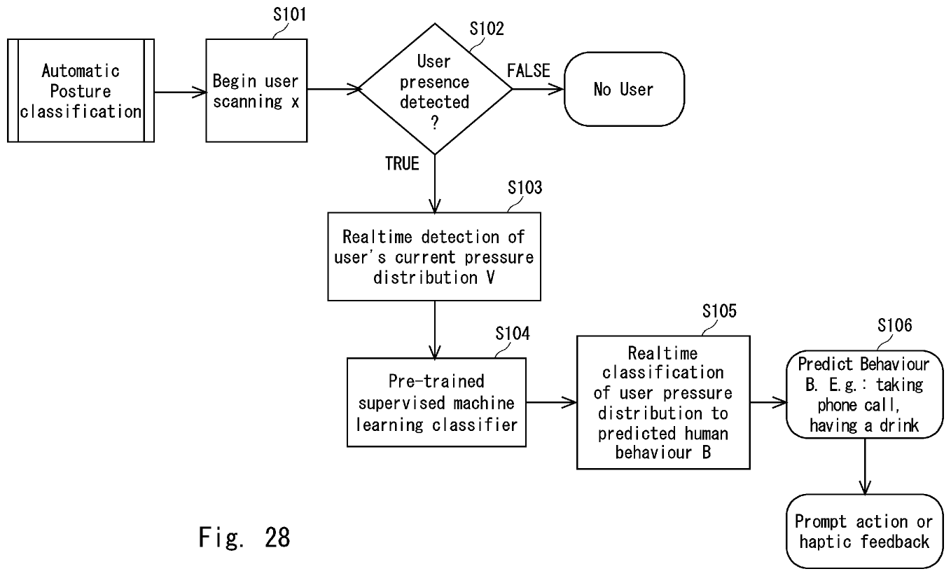

FIG. 28 is a flowchart showing a method for predicting a user behavior (action) using a machine learning model. Here, a machine learning model pre-trained on learning data is used as a classifier. For example, supervised learning is used as the learning method. The pressure distribution data for the user X is acquired in advance as the learning data. Furthermore, the user X's behavior when the pressure distribution data is acquired is associated with the training data as a correct target class label (training data). As described above, the pressure distribution data includes the detected pressure of each sensor.

The user behavior that can be classified is, for example, “taking a phone call”, “having a drink”, etc., and are defined in advance. For example, the pressure distribution data when the predefined user behavior is performed becomes the learning data. Furthermore, the user behavior is attached to the pressure distribution data, which is the learning data, as a correct answer label. The classifier is generated by performing supervised machine learning using the learning data including the correct answer label.

First, the data of the user X sitting on the chair 2 is scanned (S101). That is, the user of the user terminal 160 that has accessed the control module 102 is identified. For example, in the case of a smartphone, a user is identified for each user terminal 160. When the user terminal is, for example, a shared personal computer, the user X may be identified by a login ID, etc.

Then, the presence of the user is detected (S102). When the presence of the user has not been detected (FALSE in S102), the user is not sitting, and the process ends. When the presence of the user is detected (TRUE in S102), the pressure sensor unit 110 or the like detects a current pressure distribution V of the pressure applied from the user in real time (S103). As described above, the control module 102 acquires the detected pressure from each sensor as the current pressure distribution V.

The pressure distribution V is input to the classifier that has learned by supervised machine learning (S104). The classifier outputs a behavior label B expected from the pressure distribution V, thereby classifying a user behavior B in real time (S105). Then, the user behavior B is determined. (S106). As described above, by using the machine learning model, it is possible to appropriately classify the user behavior.

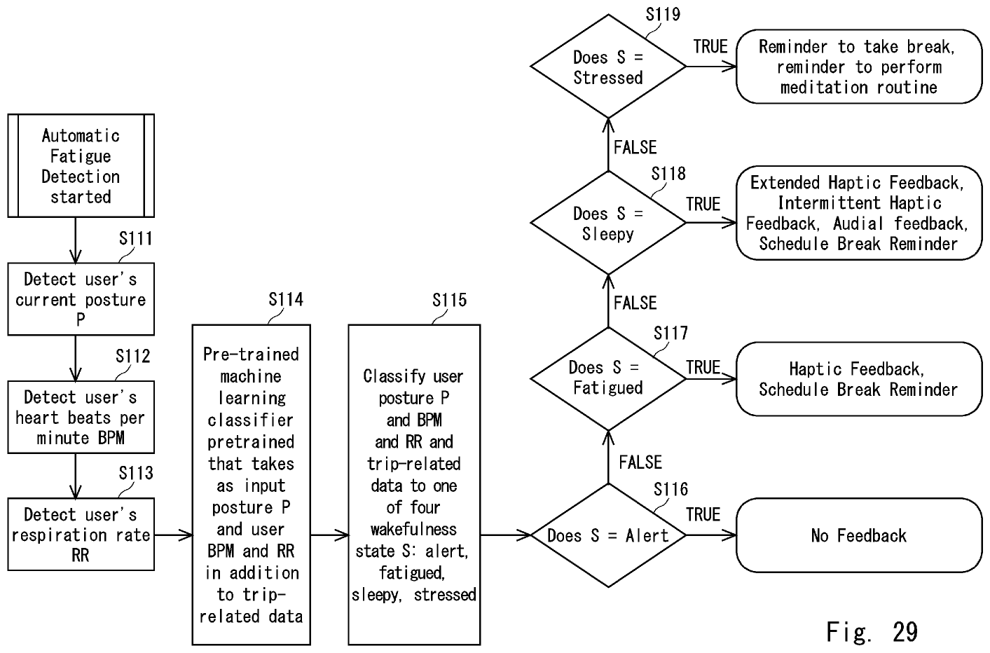

FIG. 29 is a flowchart showing a method for estimating the user's fatigue level using a machine learning model. Here, the user is driver of a vehicle and the user's fatigue level is evaluated in four stages: “alert”, “fatigued”, “sleepy”, and “stressed”. That is, the classifier classifies the user's fatigue levels into four. The machine learning model takes the user's posture P and vital information as inputs. For example, the user's fatigue level is classified by inputting the classified posture P, the heart rate (heart beats per minute BPM) and the respiration rate RR to the learned model. Further, when the user is a car driver, a trip-related data such as driving distance, driving time, average driving speed and so on may be input to the machine learning model.

First, the user's current posture P detected (S111). As described above, the posture P can be classified based on the detection data by using the table T or the learned model. Next, the vibration sensor 180 detects the user's heart beats per minute BPM (S112). The vibration sensor 180 inputs the respiration rate RR (S113). The heart beats per minute BPM and the respiration rate RR may be detected using a sensor other than the vibration sensor 180.

The posture detection system 1 inputs the posture P, the heart beats per minute BPM, and the respiration rate RR into the machine learning model (S114). When the user is a car driver, the posture detection system 1 may input the trip-related data such as the driving distance and so on to the machine learning model. The posture detection system 1 outputs the user's fatigue level S from the posture P, the heart beats per minute BPM, and the respiration rate RR using the learned model. That is, the user's fatigue level S is classified into one of four levels of “alert”, “fatigued”, “sleepy”, and “stressed” according to the learned model.

The posture detection system 1 determines whether the classified fatigue level S is “alert” (S116). When the fatigue level S is “alert” (TRUE in S116), the feedback mechanism 120 does not provide feedback. When the fatigue level S is not “alert” (FALSE in S116), the posture detection system 1 determines whether the fatigue level S is “fatigued” (S117).

When the fatigue level S is “fatigued” (TRUE in S117), the feedback mechanism 120 provides vibration feedback and outputs a reminder scheduled for a break. When the fatigue level S is not “fatigued” (FALSE in S117), the posture detection system 1 determines whether the classified fatigue level S is “sleepy” (S118).

When the fatigue level S is “sleepy” (TRUE in S118), the feedback mechanism 120 outputs extended vibration feedback, intermittent vibration feedback, audial feedback, and a reminder scheduled for a break. When the fatigue level S is not “sleepy” (FALSE in S118), the posture detection system 1 determines whether the classified fatigue level S is “stressed” (S119). When the fatigue level S is “stressed” (TRUE in S119), the feedback mechanism 120 outputs a break reminder and a meditation reminder. By doing so, the fatigue level S can be evaluated appropriately, and the feedback according to the fatigue level S can be provided.

(User Identification)

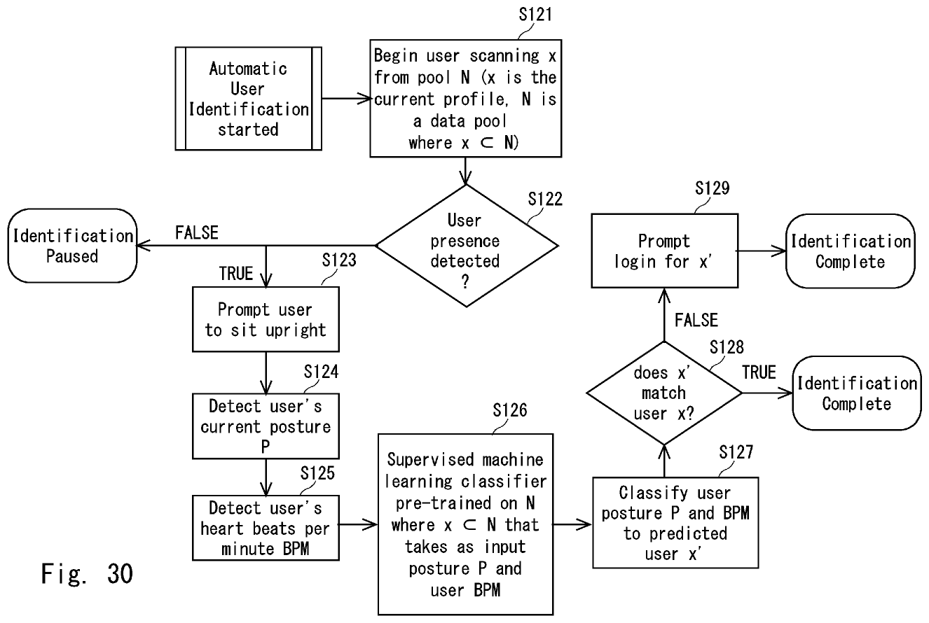

The posture detection system 1 can also identify a sitting user according to the detected pressure distribution. FIG. 30 is a flowchart showing processing for identifying a user. Here, a description will be made assuming that profile data related to N persons (N is an integer of 2 or more) is stored in advance in a pool. The profile data includes output data of each sensor at the time of calibration. That is, the detection data acquired while the user is sitting with a correct posture for calibration is the profile data.

The posture detection system 1 starts the process by identifying a user x (last logged in) whose profile is previously recorded and stored in a data pool of multiple users N (S121). A user sits on the chair 2 and the posture detection system 1 detects the a user presence (S122). When the user presence is not detected (S122 NO), the identification process is paused. When the user is presence (S122 YES), the user is prompted to sit upright (S123). For example, the user terminal displays a message or the like on the display unit 160a.

Then, the posture detection system 1 detects the user's current posture P as the upright posture based on the pressure distribution (S124). The posture detection system 1 records the detected data of the pressure distribution of this user's upright posture. Also the posture detection system 1 detects other vitals data like BPM or respiration data from the vibration sensors 180 (S125). The posture detection system 1 records the vitals data.

The combination of the upright posture pressure data and the vitals data for this user will be input into a supervised machine learning classifier that was trained on this type of data from all users in pool N (S126). The supervised machine laerning classyfier predicts user x′ from posture and BPM date and output the user profile or ID. That is, the The output will be the the user profile or ID (predict user c from posture and BPM data)

The system determines whether the predeteced user x′ matches user x or not (S128). When the predicted label or predicted user x′ profile matches the last profile login in (S128 TRUE), the identification is completed. That is, the user x′ is user x (last logged in). When the predicted label or predicted user x′ profile does not matches the last profile loged in (S128 FALSE), the system identifes user as the predicted label that is output and prompt login for that profile (user x′).

The user's current posture P is detected (S124). That is, the pressure sensor unit 110 detects the pressure distribution. Further, the vibration sensor 180 detects the user's heart beats per minute BPM (S125). Obviously, the heart beats per minute BPM may be detected by a sensor other than the vibration sensor 180. Further, the respiration rate may be used instead of the heart beats per minute BPM or together with the heart beats per minute BPM.

The current posture P and heart beats per minute BPM of the machine learning model are input (S126). The user X is predicted from the user's posture P and heart beats per minute BPM (S127). Then, it is determined whether x matches x′ (S128).

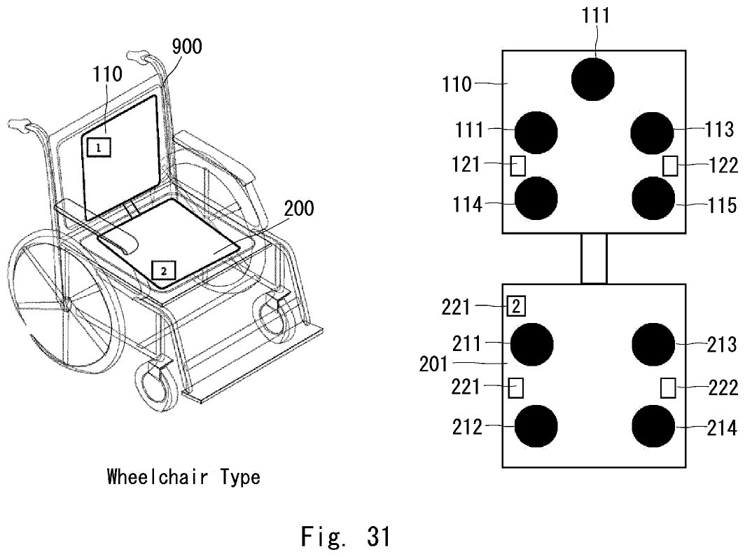

FIGS. 31 to 35 are drawings showing an example of the embodiment. FIG. 31 is a drawing showing an example in which the pressure sensor unit 110 and the seating face sensor unit 201 are mounted on a wheelchair 900. In FIG. 31, the pressure sensor unit 110 includes five sensors 111 to 115 and two vibrators 121 and 122. The seating face sensor unit 201 includes four sensors 211 to 214 and two vibrators 221 and 222. The pressure sensor unit 110 is provided in a backrest part of the wheelchair 900, and the seating face sensor unit 201 is provided in the seating face of the wheelchair 900.

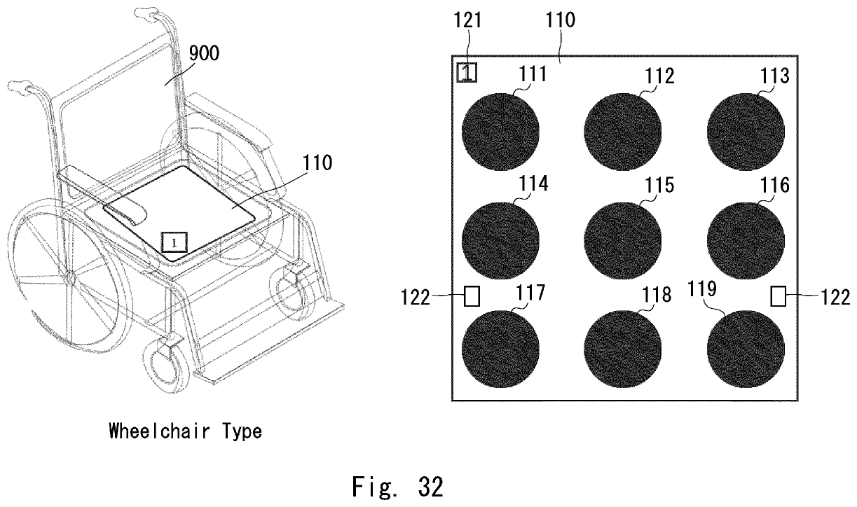

In FIG. 32, the pressure sensor unit 110 is provided in a seating face of the wheelchair 900. The pressure sensor unit 110 includes nine sensors 111 to 119 and two vibrators 121 and 122. As shown in FIG. 32, the pressure sensor 110 is not attached to the backrest of the wheelchair. In this way, the pressure sensor unit 110 may be provided in a seating face instead of the backrest part.

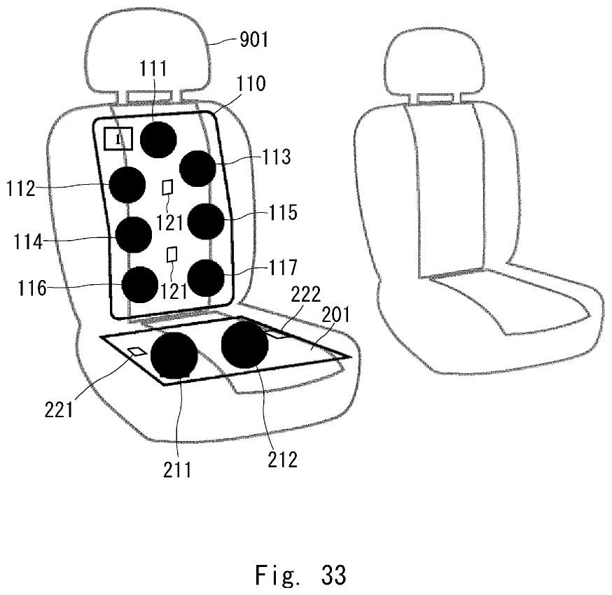

In FIG. 33, the pressure sensor unit 110 and the seating face sensor unit 201 are provided in a seat 901 of a vehicle. The pressure sensor unit 110 includes seven sensors 111 to 117 and two vibrators 121. The seating face sensor unit 201 includes two sensors 211 and 212 and two vibrators 221 and 222.

As described above, the pressure sensor unit 110 can be applied to a chair, a seat, and so forth. Thus, a user's posture can be detected appropriately.

A part or all of the processing in the embodiments may be executed by a computer program. The program can be stored and provided to a computer using any type of non-transitory computer readable media. Non-transitory computer readable media include any type of tangible storage media. Examples of non-transitory computer readable media include magnetic storage media (such as floppy disks, magnetic tapes, hard disk drives, etc.), optical magnetic storage media (e.g. magneto-optical disks), CD-ROM (Read Only Memory), CD-R, CD-R/W, and semiconductor memories (such as mask ROM, PROM (Programmable ROM), EPROM (Erasable PROM), flash ROM, RAM (random access memory), etc.). The program may be provided to a computer using any type of transitory computer readable media. Examples of transitory computer readable media include electric signals, optical signals, and electromagnetic waves. Transitory computer readable media can provide the program to a computer via a wired communication line (e.g. electric wires, and optical fibers) or a wireless communication line.

As described above, the disclosure made by the present inventor has been described in detail based on the first and second embodiments. It is obvious that the present disclosure is not limited to the above-described embodiments, and various modifications can be made without departing from the scope of the disclosure.

### REFERENCE SIGNS LIST

1 POSTURE DETECTION SYSTEM

2 CHAIR

100 BACKREST CUSHION

101 CUSHION PART

102 CONTROL MODULE

103 BELT

110 PRESSURE SENSOR UNIT

111 SENSOR

119 SENSOR

120 FEEDBACK MECHANISM

121 VIBRATOR

122 VIBRATOR

131 FIRST LAYER

132 SECOND LAYER

133 THIRD LAYER

134 FOURTH LAYER

135 FRONT COVER LAYER

136 BACK COVER LAYER

200 SEATING FACE CUSHION

201 SEATING FACE SENSOR UNIT

210 FIRST SEATING FACE SENSOR SHEET

211 TO 219 SENSOR

221 TO 222 VIBRATOR

230 SECOND SEATING FACE SENSOR SHEET

231 TO 239 SENSOR

241 VIBRATOR

242 VIBRATOR

1. A posture detection system for detecting a user's posture comprising:
a pressure sensor unit including plurality of sensors, each of the sensors being configured to detect a pressure applied from a user;
a controller configured to classify the user's posture based on detection data detected by the pressure sensor unit;
a feedback mechanism configured to provide feedback to the user by vibrating based on a result of the classification; and
a display unit configured to perform a display according to the result of the classification.

2. The posture detection system according to claim 1, wherein
the controller and the display unit are mounted on a user terminal.

3. The posture detection system according to claim 1, wherein
the pressure sensor unit is configured to detect a pressure applied from the user's back, bottom or thighs.

4. The posture detection system according to claim 1, wherein the pressure sensor unit is provided in a backrest and is configured to detect a pressure applied from the user's back.

5. The posture detection system according to claim 4, further comprising a seating face sensor unit provided in the user's seating face and configured to detect the pressure applied from the user's bottom, wherein
the controller is configured to classify the user's posture based on detection data of the seating face sensor unit.

6. The posture detection system according to claim 1, wherein
a reminder is output based on the detection data detected by the pressure sensor unit.

7. The posture detection system according to claim 1, wherein
the pressure sensor unit comprises:
a first layer including a plurality of sensing electrodes formed of conductive fabric or conductive tape;
a second layer including a conductive sheet with a variable resistance changing according to the pressure applied from the user; and
a third layer including at least one counter electrode placed to face the plurality of sensing electrodes, the counter electrode being formed of conductive fabric or conductive tape,
wherein the second layer is place between the first and third layer.

8. The posture detection system according to claim 7, wherein the sensing electrodes are formed of conductive tape,
the sensing electrode is in contact with the second layer.

9. The posture detection system according to claim 7, wherein
the pressure sensor unit further comprises a fourth layer placed between the first layer and the second layer and formed by a foam material,
the fourth layer includes a plurality of openings corresponding to the sensing electrode, respectively, and
when the pressure applied from the user exceeds a predetermined value, the sensing electrode is brought into contact with the conductive sheet through the opening.

10. The posture detection system according to claim 7, wherein
the pressure sensor unit further comprises a fourth layer placed between the second layer and the third layer,
the fourth layer includes a plurality of openings corresponding to the sensing electrode, respectively, and
when the pressure applied from the user exceeds a predetermined value, the counter electrode is brought into contact with the conductive sheet through the opening.

11. The posture detection system according to claim 1, wherein
the feedback mechanism includes a plurality of actuators for vibrating the backrest or the seating face, and
the controller is configured to operate the actuators in a pattern according to the result of the classification.

12. The posture detection system according to claim 1, wherein
each of the sensors is configured to detect, as a reference pressure, a pressure when the user is sitting with his/her back leaning against the backrest with a reference posture,
the controller is configured to calculate a difference value between the reference pressure of each of the sensors and a current pressure, and
the controller is configured to calculate a balance in a left and right direction and a balance in a vertical direction based on the difference value of each of the sensors.

13. The posture detection system according to claim 1, wherein
the display unit is configured to display a recommended pose for the user, and
it is determined whether the user's pose matches the recommended pose according to a result of the detection by the pressure sensor unit, and feedback is provided according to a result of the determination.

14. The posture detection system according to claim 13, wherein
the recommended pose is one of a stretch pose, a meditation pose, and an exercise pose.

15. The posture detection system according to claim 1, further comprising an elastic exercise member.

16. The posture detection system according to claim 15, wherein
the display unit is configured to display the exercise pose using the exercise member as the recommended pose, and
the controller is configured to determine whether the user's pose matches the recommended pose, and the feedback mechanism is configured to provide the feedback according to a result of the determination.

17. The posture detection system according to claim 1, wherein
user information about the user's physical features is input to the controller, and
the controller is configured to define the user's ideal posture based on the user information.

18. The posture detection system according to claim 1, wherein
the controller comprises a data storage unit configured to store the detection data of the pressure sensor unit for a plurality of the users, and
the controller is configured to refer to the data stored in the data storage unit and identify the user according to the result of the detection by the pressure sensor unit.

19. The posture detection system according to claim 1, further comprising a vibration sensor provided in the backrest configured to detect a vibration applied from the user, wherein
the vibration sensor is configured to detect the user's heart beats per minute or respiration rate according to a result of the detection of the vibration sensor.

20. The posture detection system according to claim 1, wherein
the controller is configured to estimate the user's fatigue level according to the result of the detection by the pressure sensor unit.

21. The posture detection system according to claim 20, wherein
when the user's fatigue level exceeds a threshold, the controller is configured to output an alert to the user.

22. The posture detection system according to claim 1, wherein
the feedback mechanism is configured to vibrate periodically.

23. The posture detection system according to claim 1, wherein
when the posture classified by the controller continues for a predetermined period or longer, the feedback mechanism is configured to provide the feedback by the vibration.

24. The posture detection system according to claim 1, wherein
the display unit is configured to display a report including at least one of:
summary of the sedentary performance or activeness,
a score of the posture or activeness, wherein the score of the posture or activeness is determined for a time period based on at least one of a sitting time duration, a percentage of occurrence of the posture, a frequency of breaks, a duration of breaks, pressure distribution symmetry value and a detection of performing stretches and
recommended action including a stretch pose, exercise routine or a sedentary guidance, wherein the stretch pose is associated with the classified posture and wherein the sedentary guidance is classified based on a history of the user's postures and a score of the posture or activeness of the user.

25. The posture detection system according to claim 1, wherein
the controller classifies the posture by a machine learning tool,
input data of the supervised machine learning tool includes information of physical feature of the user and detection date of the pressure sensor unit.

26. The posture detection system according to claim 1, wherein
a pressure distribution is measured by detection data of the pressure sensor unit,
a behavior of the user is estimated by a machine learning tool,
input data of the supervised machine learning tool includes information of physical feature of the user, the detection data of the pressure sensor unit, a score of the posture or activeness, and the time of the day.

27. The posture detection system according to claim 1, wherein
the user's state is predicted according to a result of the prediction of the user's behavior.

28. A posture detection method for detecting a user's posture, the posture detection method comprising:
detecting a pressure applied from a user using a pressure sensor unit, the pressure sensor unit including a sheet shape or a padded shape and including plurality of sensors, and each of the sensors being configured to detect the pressure applied from the user;
classifying the user's posture based on detection data detected by the pressure sensor unit;
providing feedback to the user by vibrating based on a result of the classification; and
performing a display according to the result of the classification.
